SEQUENCE LISTING (16 sequences)

SEQ ID NO: 1 - DNA, 5756 bp, Homo sapiens
gttttactaaagtgaatttttttttgtttgcttcgttcgtctttggctctttttttttccttcccaatttcggatttatttcaaggcgaatctggctttgggggaagaggaagaaaagtcggattacaagatcaaccaccaccaacaacaataaaaaccaccaggatatttttttgcaaatttctgacggctttaaattcatgaagcaattgtccccttttgcaatcagcatttggatctcagaatgagcaaggaaagacccaagaggaatatcattcagaagaaatacgatgacagtgatgggattccgtggtcagaagaacgggtggtacgtaaagtcctttatttgtctctgaaggagttcaagaattcccagaagaggcagcatgcggaaggcattgctgggagcctgaaaactgtgaatgggctccttggtaatgaccagtctaagggattaggaccagcatcagaacagtcagagaatgaaaaggacgatgcatcccaagtgtcctccactagcaacgatgttagttcttcagattttgaagaagggccgtcgaggaaaaggcccaggctgcaagcacaaaggaagtttgctcagtctcagccgaatagtcccagcacaactccagtaaagatagtggagccattgctaccccctccagctactcagatatcagacctctctaaaaggaagcctaagacagaagattttcttacctttctctgccttcgaggttctcctgcgctgcccaacagcatggtgtattttggaagctctcaggatgaggaggaagtcgaggaggaagatgatgagacagaagacgtcaaaacagccaccaacaatgcttcatcttcatgccagtcgacccccaggaaaggaaaaacccacaaacatgttcacaacgggcatgttttcaatggttccagcaggtcaacacgggagaaggaacctgttcaaaaacacaaaagcaaagaggccactcccgcaaaggagaagcacagcgatcaccgggctgacagccgccgggagcaggcttcagctaaccaccccgcagcggccccctccacgggttcctcggccaaggggcttgctgccacccatcaccacccccctctgcatcggtcggctcaggacttacggaaacaggtttctaaggtaaacggagtcactcgaatgtcatctctgggtgcaggtgtaaccagtgccaaaaagatgcgcgaggtcagaccttcaccatccaaaactgtgaagtacactgccacggtgacgaagggggctgtcacatacaccaaagccaagagagaactggtcaaggacaccaaacccaatcaccacaagcccagttccgctgtcaaccacacaatctcagggaaaactgaaagtagcaatgcaaaaacccgcaaacaggtgctatccctcgggggggcgtccaagtccactgggcccgccgtcaatggcctcaaggtcagtggcaggttgaacccaaagtcatgcactaaggaggtgggggggcggcagctgcgggagggcctgcagctgcgggaggggctgcggaactccaagaggagactggaagaggcacaccaggcggagaagccgcagtcgccccccaagaagatgaaaggggcggctggccccgccgaaggccctggcaagaaggccccggccgagagaggtctgctgaacggacacgtgaagaaggaagtgccggagcgcagtctggagaggaatcggccgaagcgggccacggccgggaagagcacgccaggcagacaagcacatggcaaggcggacagcgcctcctgtgaaaatcgttctacctcgcaaccggagtccgtgcacaagccgcaggactcgggcaaggccgagaagggcggcggcaaggccgggtgggcggccatggacgagatccccgtcctcaggccctccgccaaggagttccacgatccgctcatctacatcgagtcggtccgcgctcaggtggagaagttcgggatgtgcagggtgatcccccctccggactggcggcccgagtgcaagctcaacgatgagatgcggtttgtcacgcagattcagcacatccacaagctgggccggcgctggggccccaacgtgcagcggctggcctgcatcaagaagcacctcaaatctcagggcatcaccatggacgagctcccgctcatagggggctgtgagctcgacctggcctgctttttccggctgattaatgagatgggcggcatgcagcaagtgactgacctcaaaaaatggaacaaactagcagacatgctgcgcatccccagaactgcccaggaccggctggccaagctgcaggaggcctactgccagtacctactctcctacgactccctgtccccagaggagcaccggcggctggagaaggaggtgctgatggagaaggagatcctggagaagcgcaaggggccgctggaaggccacacagagaacgaccaccacaagttccaccctctgccccgcttcgagcccaagaatgggctcatccacggcgtggcccccaggaacggcttccgcagcaagctcaaggaggtgggccaggcccagttgaagactggccggcggcgactcttcgctcaggaaaaagaagtggtcaaggaagaggaggaggacaaaggcgtcctcaatgacttccacaagtgcatctataagggaaggtctgtttctctaacaactttttatcgaacagcgaggaatatcatgagcatgtgtttcagcaaggagcctgccccagccgaaatcgagcaagagtactggaggctagtggaagagaaggactgccacgtggcagtgcactgcggcaaggtggacaccaacactcacggcagtggattcccagtaggaaaatcagaacccttttcgaggcatggatggaacctcaccgtcctccccaataacacagggtccatcctgcgtcacctcggtgctgtgcctggagtgactattccctggctaaatattggcatggtcttttctacctcatgctggtctcgagaccaaaatcaccttccatacattgactacttacacactggtgctgactgcatttggtattgcattcctgctgaggaggagaacaagctggaagatgtggtccacaccctgctgcaagccaatggcaccccagggctgcagatgctggaaagcaacgtcatgatctccccggaggtgctgtgcaaagaggggatcaaggtgcacaggaccgtgcagcagagtggccagtttgtcgtctgcttcccgggatcctttgtgtccaaagtgtgctgtgggtacagcgtgtctgaaaccgtgcactttgctaccacccagtggacaagtatgggctttgagaccgccaaggaaatgaagcgtcgccatatagctaagccattctccatggagaagttactctaccagattgcacaagcagaagcaaaaaaagaaaacggtcccactctcagtaccatctcagccctcctggatgagctcagggatacagagctgcggcagcgcaggcagctgttcgaggctggcctccactcctccgcacgctatggcagccacgatggcagcagcacggtggcggacgggaagaaaaagcctcgaaagtggctgcagttggagacgtcagagaggaggtgtcagatctgccagcacctgtgctacctgtccatggtggtacaagagaacgaaaacgtcgtgttctgtctggagtgtgctctgcgccacgtggagaaacagaagtcctgccgagggctgaagttgatgtaccgctacgatgaggaacagattatcagtctggtcaatcagatctgcggcaaagtgtctggtaaaaacggcagcattgagaactgtctcagtaaacccacaccaaaaagaggtccccgcaagagagcgacagtggacgtgcccccctcccgtctgtcagcctccagttcatccaaaagtgcttcgagctcatcatgaagatgccaacgcccgtggtcgatttatatatatttttttgtaattattatattctagtttggagtacttgctgtaggattcaagctgtctttgcactagctctaaagaagattttcttctggttttagagaactaattttgttttagcattaaactgttgaacttttttttgtacttagaaaacctagatactgcagtcagattttggaaactgccgtatagtcactgttttaaaaaccccggaggggctgtattaatttgtattgccccatggctgacaaaagcctttttttttggttttgatttttttttttttgtaactgttggggggaaaaaggctttttaacccatttttgaagagggtgaagtttggagaacaaatttaaaaaccatcagtcatgtgagcagattttttagaagggataggagacacacgcgcacacacacacacacacgaaacttgaaatggctttgctttggctgtcgtcttctgccgtgtgccagatgagcttgtgatctgggaagccggggcacccccgttttgtttctctgggcggttgtggcagctgaaggcggacgttgtttcctaaccataggtggaacgaggagacgggagcgagtgggctctccaccagcacatcactatgcatctgttccaggaaagaagaaaagcgagcgaggaagacggaaaagactgcctgccttggaggggtcacatgagggagacctgtgcctgatttcattaggaaatccattctgttattttttggtgctgttggctactttatcaaaaaacccttcaatagcatccttaagatttaaaaaaaaaaaaaaaaaaaaggaaaaaaaagtgatggaagccgtaagtgcttctttgtcatcgacgtgcaatctttctaacattccatctccatctcaccgcttcttgtttgacaccttcacaagtcagcattaatctttcttttaaaacttgtttcatttatgatcatgtagagagccactaggaggcctgcagttatttttgaatgtgaaaatgcatttgcgttcatcttgtctattttttctcttcatgttgtaacaaaaaggaaaaaagaaaaaaaaatcccatcccttttgtacatatgcctgtaaattgttttaaatacttgagcctttttctcggtggggggtggggaggggggtgagaagacaagatgaagaaaagccttacatttcagtttcttcatcggttggattggatgcttacagggtttttcttgtaacatttataagtgctgcttacatcactgaacaacaacaaaaaaataataatggagtagctgttgcccttctccggttgtgtgtacagtatgtgtggaataaaaaagggaaactgttttcacaagctgttctttgtttcataattggattcatcaatcccgtagctacccatattgcactgagcttgccagtggtgactgccaggaacgtcctatgatccactttgttggttgttgttgcagaagactgaactgttttggaatatttaacaattacagaaacagtcaagtgttttccaatgtggttgtccggtttctatggccttgctgtgtactttccctctttttgacagtaaacttctgcctatggcttacagtttgacatttaatttattagcgctgctctgcacccctcccttgggagggagacttcatgtggtttattgcgagttttttgtttacttttcaggtttgtactacaaggtttaataataaaaacaaagttttttgga

SEQ ID NO: 2 - DNA, 52 bp, Homo sapiens
cccagagggtttatattttacctgcaytcctgaggatgtgtttgtgttgctt

SEQ ID NO: 3 - DNA, 52 bp, Homo sapiens
atgttctttcaatggaagcccccaccytctgagtacactggttcatagttat

SEQ ID NO: 4 - DNA, 52 bp, Homo sapiens
gcgaatgcatttgttgggattgacttyaataatgaggctggttttgtttaaa

SEQ ID NO: 5 - DNA, 52 bp, Homo sapiens
tgtccttgagaaactcataagttgcartgtaatcctgtcttaattgtgttga

SEQ ID NO: 6 - DNA, 52 bp, Homo sapiens
aaagaggaaccctactggtagaagttyttgagagctattcttgagagctggt

SEQ ID NO: 7 - DNA, 52 bp, Homo sapiens
tgcaaaaaagggacagtcagattaaamtgtggacagcagagtagttgttcat

SEQ ID NO: 8 - DNA, 52 bp, Homo sapiens
aacctctgttttgttgggttacctccrtctctgtgacttggggtgacaacct

SEQ ID NO: 9 - DNA, 52 bp, Homo sapiens
cccttccctcactgactttatatttckggaaatttcatgtctagggaagttg

SEQ ID NO: 10 - DNA, 52 bp, Homo sapiens
gagatgcagcttccagtcagtgcgcayataccacttggagggcatgctggtt

SEQ ID NO: 11 - DNA, 52 bp, Homo sapiens
ttggagggcatgctggttgcaaccctyttattctaataaggaactggtttgg

SEQ ID NO: 12 - DNA, 52 bp, Homo sapiens
aggatgaggtgagcttaccaaccccamtgagtaggggccaaacatccttaac

SEQ ID NO: 13 - DNA, 52 bp, Homo sapiens
aacatgatttccttcagcttctcctcrtatttacaagccaattgcttgactc

SEQ ID NO: 14 - DNA, 52 bp, Homo sapiens
aaacatctcaaaactgcacagaagaayccatcaaaaaattttatgtaacagt

SEQ ID NO: 15 - DNA, 52 bp, Homo sapiens
cttacgttttcttagagttacatgggmaacattgtagttcagcacagccctt

SEQ ID NO: 16 - DNA, 52 bp, Homo sapiens
aatccaaatgcccgtgcaggaaccccrctccccctgggatcctaacatgggg

(19) United States
(12) Patent Application Publication
(10) Pub. No.: US 2014/0127685 A1
(43) Pub. Date: May 8, 2014

(54) COMPOSITIONS AND METHODS FOR DIAGNOSING AUTISM

(71) Applicants: Aravinda Chakravarti, Lutherville, MD (US); Dan E. Arking, Baltimore, MD (US); Mark Daly, Boston, MA (US); Jerome Carayol, Montrouge (FR); Francis Rousseau, Savigny Sur Orge (FR)

(72) Inventors: Aravinda Chakravarti, Lutherville, MD (US); Dan E. Arking, Baltimore, MD (US); Mark Daly, Boston, MA (US); Jerome Carayol, Montrouge (FR); Francis Rousseau, Savigny Sur Orge (FR)

(73) Assignees: The Johns Hopkins University, Baltimore, MD (US); The General Hospital Corporation, Boston, MA (US); IntegraGen SA, Evry Cedex (FR)

(21) Appl. No.: 13/878,194

(22) PCT Filed: Oct. 8, 2010
(86) PCT No.: PCT/US10/52060

§ 371 Date: Jan. 15, 2014

Related U.S. Application Data
(60) Provisional application No. 61/391,035, filed on Oct. 7, 2010.

(51) Int. Cl. C12Q 1/68

(52) U.S. Cl. CPC C12Q 1/6883
USPC 435/6.11

(57) ABSTRACT

The invention provides methods featuring the use of polymorphisms in the JARID2 gene to diagnosis autism.

### CROSS-REFERENCE TO RELATED APPLICATION

This application claims the benefit of the following U.S. Provisional Application No. 61/391,035, filed Oct. 7, 2010, the entire contents of which are incorporated herein by reference.

### STATEMENT OF RIGHTS TO INVENTIONS MADE UNDER FEDERALLY SPONSORED RESEARCH

This work was supported by the following grants from the National Institutes of Health, Grant No. MH060007. The government has certain rights in the invention.

### BACKGROUND OF THE INVENTION

Autistic spectrum disorders (MIM 209850) are a group of neurodevelopment disorders that include autism, Asperger syndrome, and pervasive developmental disorder not otherwise specified (PDD-NOS). These disorders are characterized by impairment in communications and social interactions and the presence of stereotypical behaviors. The sex-ratio of autism is 4 to 1 male to female, and the prevalence of the disease is estimated to be above 1 per 100 persons. The etiology of autistic spectrum disorders is unknown, but family and twin studies have shown a high monozygotic and dizygotic twin risk ration and a sib relative risk between 50 and 100, indicating that the predisposition to develop autism is largely genetically determined.

Although autism is a highly heritable neurodevelopmental disorder, attempts to identify specific susceptibility genes have thus far met with limited success. Autism genes have been difficult to identify, despite the high heritability of autism spectrum disorders. Up to 10% of autism cases may be due to rare sequence and gene dosage variants, for example, mutations in NRXN1, NLGN3/4X, SHANK3, and copy number variants at 15q1 1-q1 3 and 16p11. 2. A number of diseases of known etiology, including Rett syndrome, fragile X syndrome, neurofibromatosis type I, tuberous sclerosis, Potocki-Lupski syndrome, and Smith-Lemli-Opitz syndrome are also associated with autism (Abrahams and Geschwind 2008; Zafeiriou et al. 2007). However, the remaining 90% of autism spectrum disorders, while highly familial, have unknown genetic etiology. Genome-wide association studies using half a million or more markers, particularly those with very large sample sizes achieved through meta-analysis, have shown great success in mapping genes for other complex genetic traits.

Early diagnosis and intervention can ameliorate many symptoms of autism. Such intervention is most effective when infants and toddlers are developing language and social skills. Presently, most children with autism wait until they are about five years old for a definitive diagnosis. At this point, they are well past the age when intervention is most effect. An urgent need exists for genetic markers and methods of using such markers to identify infants and toddlers as having or having a propensity to develop autism. Such markers would also elucidate the molecular mechanisms that are the root causes of the disease.

### SUMMARY OF THE INVENTION

The invention provides genetic alterations associated with autism and autism spectrum disorders and methods of using such markers for the diagnosis of subjects having or having a propensity to develop autism and autism-related disorders.

In one aspect, the invention generally features a method for determining a genetic predisposition to or for the presence of autism or an autism spectrum disorder in a subject, the method involving identifying the presence of absence of a genetic alteration in a JARID2 nucleic acid molecule derived from the subject.

In another aspect, the invention generally features a method for identifying a subject as in need of therapeutic intervention to ameliorate autism or an autism spectrum disorder, the method involving identifying the presence or absence of a genetic alteration in a JARID2 nucleic acid molecule derived from the subject.

In another aspect, the invention generally features a kit for detecting an autism-associated polymorphism in a subject, the kit comprising at least one set of primers suitable for use in polymerase chain reaction (PCR), wherein the set of primers amplifies a JARID2 nucleic acid molecule.

In another aspect, the invention features a kit for detecting an autism-associated polymorphism in a subject, the kit comprising at least one polynucleotide molecule capable of specifically binding or hybridizing to a polymorphism in a JARID2 nucleic acid molecule and directions for using the kit.

In another aspect, the invention generally features a kit for detecting an autism associated polymorphism in a subject, the kit comprising at least one set of primers suitable for use in polymerase chain reaction (PCR), wherein the set of primers amplifies polymorphism site selected from the group consisting of rs7766973, rs6915344, rs12530202, rs2295954, rs9464779, rs11962776, rs6921502, rs9396578, rs6459404, rs9370809, rs3759, rs957387, rs707833, rs13193457, rs909626.

In various embodiments of any of the above aspects or any other aspect of the invention delineated herein, the subject is identified as having a family member diagnosed with autism. In another embodiment the subject is identified as having a family member diagnosed with schizophrenia. In yet other embodiments the genetic alteration is in a linkage disequilibrium region of JARID2 or is associated with chromosome 6p23. In further embodiments the genetic alteration is a single nucleotide polymorphism (SNP) in said JARID2 nucleic acid molecule. In other embodiments the SNP is selected from the group consisting of rs7766973, rs6459404, rs6921502, rs6915344, and rs13193457. In a further embodiment the identification of a C at polymorphism site rs7766973, indicates an increased risk for autism. In yet a further embodiment the genetic alteration is identified in a biological sample from the subject. In another embodiment the biological sample is selected from blood, urine, feces, saliva, a cheek swab, amniotic fluid, and tissue. In other embodiments the biological sample is blood. In yet other embodiments the sample is isolated from a subject that is between 0 and 6 months of age, between 6 and 12 months of age, or between 12 and 36 months of age. In additional embodiments the subject is a child identified as having delayed communication skills, social skills, or that is otherwise identified as developmentally disabled.

In various embodiments of any of the above aspects or any other aspect of the invention delineated herein, the method further involves comparing the genetic alteration in the subject with a corresponding sequence in a relative of the subject. In another embodiment the genetic alteration is detected by a method selected from the group consisting of direct sequencing, single strand polymorphism assay, denaturing high performance liquid chromatography, hybridization on a nucleic acid array, restriction length polymorphism assay, ligase chain reaction, enzymatic cleavage, southern hybridization, mass spectrometry, and polymerase chain reaction. In another embodiment the biological sample comprises deoxyribonucleic acid or ribonucleic acid. In additional embodiments the genetic alteration is detected by single strand polymorphism assay. In yet another embodiment the genetic alteration is detected using denaturing high performance liquid chromatography. In an additional embodiment the testing of the sample is carried out by direct sequencing of nucleic acids. In yet additional embodiments the polymorphism is at a site selected from the group consisting of rs7766973, rs6915344, rs12530202, rs2295954, rs9464779, rs11962776, rs6921502, rs9396578, rs6459404, rs9370809, rs3759, rs957387, rs707833, rs13193457, rs909626.

In various embodiments of any of the above aspects or any other aspect of the invention delineated herein, the method further involves identifying the subject as having a developmental delay or behavioral abnormality characteristic of autism.

### DEFINITIONS

By “autism” is meant a developmental disorder characterized by impaired social interaction and communication, and by restricted and repetitive behavior. Autism as used herein includes all of the disorders recognized in the autism spectrum of diseases (ASD) and thus includes Asperger Syndrome and Pervasive Developmental Disorder-Not Otherwise Specified (PDD-NOS).

By “JARID2 nucleic acid molecule” is meant a polynucleotide or fragment thereof encoding or modulating the expression of a Jarid2 polypeptide. In one embodiment, a JARID2 nucleic acid molecule is the human gene JARID2, which is an ortholog of the mouse jumonji gene. JARID2 corresponds to a polynucleotide comprising or consisting essentially of human chromosome 6:15, 354, 506 or an autism-associated genetic alteration present at gene map locus 6p23. In another embodiment, Jarid2 nucleic acid molecule comprises or consists essentially of the sequence provided at NCBI Accession No. NM_{—}004973.2. An exemplary JARID2 nucleic acid molecule sequence (i.e., NM_{—}004973.2) follows:

| | |
|---|---|
| 1 | gttttactaa agtgaatttt tttttgtttg cttcgttcgt |
| | ctttggctct ttttttttcc |
| 61 | ttcccaattt cggatttatt tcaaggcgaa tctggctttg |
| | ggggaagagg aagaaaagtc |
| 121 | ggattacaag atcaaccacc accaacaaca ataaaaacca |
| | ccaggatatt tttttgcaaa |
| 181 | tttctgacgg ctttaaattc atgaagcaat tgtccccttt |
| | tgcaatcagc atttggatct |
| 241 | cagaatgagc aaggaaagac ccaagaggaa tatcattcag |
| | aagaaatacg atgacagtga |
| 301 | tgggattccg tggtcagaag aacgggtggt acgtaaagtc |
| | ctttatttgt ctctgaagga |
| 361 | gttcaagaat tcccagaaga ggcagcatgc ggaaggcatt |
| | gctgggagcc tgaaaactgt |
| 421 | gaatgggctc cttggtaatg accagtctaa gggattagga |
| | ccagcatcag aacagtcaga |
| 481 | gaatgaaaag gacgatgcat cccaagtgtc ctccactagc |
| | aacgatgtta gttcttcaga |
| 541 | ttttgaagaa gggccgtcga ggaaaaggcc caggctgcaa |
| | gcacaaagga agtttgctca |
| 601 | gtctcagccg aatagtccca gcacaactcc agtaaagata |
| | gtggagccat tgctaccccc |
| 661 | tccagctact cagatatcag acctctctaa aaggaagcct |
| | aagacagaag attttcttac |
| 721 | ctttctctgc cttcgaggtt ctcctgcgct gcccaacagc |
| | atggtgtatt ttggaagctc |
| 781 | tcaggatgag gaggaagtcg aggaggaaga tgatgagaca |
| | gaagacgtca aaacagccac |
| 841 | caacaatgct tcatcttcat gccagtcgac ccccaggaaa |
| | ggaaaaaccc acaaacatgt |
| 901 | tcacaacggg catgttttca atggttccag caggtcaaca |
| | cgggagaagg aacctgttca |
| 961 | aaaacacaaa agcaaagagg ccactcccgc aaaggagaag |
| | cacagcgatc accgggctga |
| 1021 | cagccgccgg gagcaggctt cagctaacca ccccgcagcg |
| | gccccctcca cgggttcctc |
| 1081 | ggccaagggg cttgctgcca cccatcacca cccccctctg |
| | catcggtcgg ctcaggactt |
| 1141 | acggaaacag gtttctaagg taaacggagt cactcgaatg |
| | tcatctctgg gtgcaggtgt |
| 1201 | aaccagtgcc aaaaagatgc gcgaggtcag accttcacca |
| | tccaaaactg tgaagtacac |
| 1261 | tgccacggtg acgaaggggg ctgtcacata caccaaagcc |
| | aagagagaac tggtcaagga |
| 1321 | caccaaaccc aatcaccaca agcccagttc cgctgtcaac |
| | cacacaatct cagggaaaac |
| 1381 | tgaaagtagc aatgcaaaaa cccgcaaaca ggtgctatcc |
| | ctcggggggg cgtccaagtc |
| 1441 | cactgggccc gccgtcaatg gcctcaaggt cagtggcagg |
| | ttgaacccaa agtcatgcac |
| 1501 | taaggaggtg ggggggcggc agctgcggga gggcctgcag |
| | ctgcgggagg ggctgcggaa |
| 1561 | ctccaagagg agactggaag aggcacacca ggcggagaag |
| | ccgcagtcgc cccccaagaa |
| 1621 | gatgaaaggg gcggctggcc ccgccgaagg ccctggcaag |
| | aaggccccgg ccgagagagg |
| 1681 | tctgctgaac ggacacgtga agaaggaagt gccggagcgc |
| | agtctggaga ggaatcggcc |
| 1741 | gaagcgggcc acggccggga agagcacgcc aggcagacaa |
| | gcacatggca aggcggacag |
| 1801 | cgcctcctgt gaaaatcgtt ctacctcgca accggagtcc |
| | gtgcacaagc cgcaggactc |
| 1861 | gggcaaggcc gagaagggcg gcggcaaggc cgggtgggcg |
| | gccatggacg agatccccgt |
| 1921 | cctcaggccc tccgccaagg agttccacga tccgctcatc |
| | tacatcgagt cggtccgcgc |
| 1981 | tcaggtggag aagttcggga tgtgcagggt gatcccccct |
| | ccggactggc ggcccgagtg |
| 2041 | caagctcaac gatgagatgc ggtttgtcac gcagattcag |
| | cacatccaca agctgggccg |
| 2101 | gcgctggggc cccaacgtgc agcggctggc ctgcatcaag |
| | aagcacctca aatctcaggg |
| 2161 | catcaccatg gacgagctcc cgctcatagg gggctgtgag |
| | ctcgacctgg cctgcttttt |
| 2221 | ccggctgatt aatgagatgg gcggcatgca gcaagtgact |
| | gacctcaaaa aatggaacaa |
| 2281 | actagcagac atgctgcgca tccccagaac tgcccaggac |
| | cggctggcca agctgcagga |
| 2341 | ggcctactgc cagtacctac tctcctacga ctccctgtcc |
| | ccagaggagc accggcggct |
| 2401 | ggagaaggag gtgctgatgg agaaggagat cctggagaag |
| | cgcaaggggc cgctggaagg |
| 2461 | ccacacagag aacgaccacc acaagttcca ccctctgccc |
| | cgcttcgagc ccaagaatgg |
| 2521 | gctcatccac ggcgtggccc ccaggaacgg cttccgcagc |
| | aagctcaagg aggtgggcca |
| 2581 | ggcccagttg aagactggcc ggcggcgact cttcgctcag |
| | gaaaaagaag tggtcaagga |
| 2641 | agaggaggag gacaaaggcg tcctcaatga cttccacaag |
| | tgcatctata agggaaggtc |
| 2701 | tgtttctcta acaacttttt atcgaacagc gaggaatatc |
| | atgagcatgt gtttcagcaa |
| 2761 | ggagcctgcc ccagccgaaa tcgagcaaga gtactggagg |
| | ctagtggaag agaaggactg |
| 2821 | ccacgtggca gtgcactgcg gcaaggtgga caccaacact |
| | cacggcagtg gattcccagt |
| 2881 | aggaaaatca gaaccctttt cgaggcatgg atggaacctc |
| | accgtcctcc ccaataacac |
| 2941 | agggtccatc ctgcgtcacc tcggtgctgt gcctggagtg |
| | actattccct ggctaaatat |
| 3001 | tggcatggtc ttttctacct catgctggtc tcgagaccaa |
| | aatcaccttc catacattga |
| 3061 | ctacttacac actggtgctg actgcatttg gtattgcatt |
| | cctgctgagg aggagaacaa |
| 3121 | gctggaagat gtggtccaca ccctgctgca agccaatggc |
| | accccagggc tgcagatgct |
| 3181 | ggaaagcaac gtcatgatct ccccggaggt gctgtgcaaa |
| | gaggggatca aggtgcacag |
| 3241 | gaccgtgcag cagagtggcc agtttgtcgt ctgcttcccg |
| | ggatcctttg tgtccaaagt |
| 3301 | gtgctgtggg tacagcgtgt ctgaaaccgt gcactttgct |
| | accacccagt ggacaagtat |
| 3361 | gggctttgag accgccaagg aaatgaagcg tcgccatata |
| | gctaagccat tctccatgga |
| 3421 | gaagttactc taccagattg cacaagcaga agcaaaaaaa |
| | gaaaacggtc ccactctcag |
| 3481 | taccatctca gccctcctgg atgagctcag ggatacagag |
| | ctgcggcagc gcaggcagct |
| 3541 | gttcgaggct ggcctccact cctccgcacg ctatggcagc |
| | cacgatggca gcagcacggt |
| 3601 | ggcggacggg aagaaaaagc ctcgaaagtg gctgcagttg |
| | gagacgtcag agaggaggtg |
| 3661 | tcagatctgc cagcacctgt gctacctgtc catggtggta |
| | caagagaacg aaaacgtcgt |
| 3721 | gttctgtctg gagtgtgctc tgcgccacgt ggagaaacag |
| | aagtcctgcc gagggctgaa |
| 3781 | gttgatgtac cgctacgatg aggaacagat tatcagtctg |
| | gtcaatcaga tctgcggcaa |
| 3841 | agtgtctggt aaaaacggca gcattgagaa ctgtctcagt |
| | aaacccacac caaaaagagg |
| 3901 | tccccgcaag agagcgacag tggacgtgcc cccctcccgt |
| | ctgtcagcct ccagttcatc |
| 3961 | caaaagtgct tcgagctcat catgaagatgc caacgcccg |
| | tggtcgattt atatatattt |
| 4021 | ttttgtaatt attatattct agtttggagt acttgctgta |
| | ggattcaagc tgtctttgca |
| 4081 | ctagctctaa agaagatttt cttctggttt tagagaacta |
| | attttgtttt agcattaaac |
| 4141 | tgttgaactt ttttttgtac ttagaaaacc tagatactgc |
| | agtcagattt tggaaactgc |
| 4201 | cgtatagtca ctgttttaaa aaccccggag gggctgtatt |
| | aatttgtatt gccccatggc |
| 4261 | tgacaaaagc cttttttttt ggttttgatt tttttttttt |
| | tgtaactgtt ggggggaaaa |
| 4321 | aggcttttta acccattttt gaagagggtg aagtttggag |
| | aacaaattta aaaaccatca |
| 4381 | gtcatgtgag cagatttttt agaagggata ggagacacac |
| | gcgcacacac acacacacac |
| 4441 | gaaacttgaa atggctttgc tttggctgtc gtcttctgcc |
| | gtgtgccaga tgagcttgtg |
| 4501 | atctgggaag ccggggcacc cccgttttgt ttctctgggc |
| | ggttgtggca gctgaaggcg |
| 4561 | gacgttgttt cctaaccata ggtggaacga ggagacggga |
| | gcgagtgggc tctccaccag |
| 4621 | cacatcacta tgcatctgtt ccaggaaaga agaaaagcga |
| | gcgaggaaga cggaaaagac |
| 4681 | tgcctgcctt ggaggggtca catgagggag acctgtgcct |
| | gatttcatta ggaaatccat |
| 4741 | tctgttattt tttggtgctg ttggctactt tatcaaaaaa |
| | cccttcaata gcatccttaa |
| 4801 | gatttaaaaa aaaaaaaaaa aaaaaggaaa aaaaagtgat |
| | ggaagccgta agtgcttctt |
| 4861 | tgtcatcgac gtgcaatctt tctaacattc catctccatc |
| | tcaccgcttc ttgtttgaca |
| 4921 | ccttcacaag tcagcattaa tctttctttt aaaacttgtt |
| | tcatttatga tcatgtagag |
| 4981 | agccactagg aggcctgcag ttatttttga atgtgaaaat |
| | gcatttgcgt tcatcttgtc |
| 5041 | tattttttct cttcatgttg taacaaaaag gaaaaaagaa |
| | aaaaaaatcc catccctttt |
| 5101 | gtacatatgc ctgtaaattg ttttaaatac ttgagccttt |
| | ttctcggtgg ggggtgggga |
| 5161 | ggggggtgag aagacaagat gaagaaaagc cttacatttc |
| | agtttcttca tcggttggat |
| 5221 | tggatgctta cagggttttt cttgtaacat ttataagtgc |
| | tgcttacatc actgaacaac |
| 5281 | aacaaaaaaa taataatgga gtagctgttg cccttctccg |
| | gttgtgtgta cagtatgtgt |
| 5341 | ggaataaaaa agggaaactg ttttcacaag ctgttctttg |
| | tttcataatt ggattcatca |
| 5401 | atcccgtagc tacccatatt gcactgagct tgccagtggt |
| | gactgccagg aacgtcctat |
| 5461 | gatccacttt gttggttgtt gttgcagaag actgaactgt |
| | tttggaatat ttaacaatta |
| 5521 | cagaaacagt caagtgtttt ccaatgtggt tgtccggttt |
| | ctatggcctt gctgtgtact |
| 5581 | ttccctcttt ttgacagtaa acttctgcct atggcttaca |
| | gtttgacatt taatttatta |
| 5641 | gcgctgctct gcacccctcc cttgggaggg agacttcatg |
| | tggtttattg cgagtttttt |
| 5701 | gtttactttt caggtttgta ctacaaggtt taataataaa |
| | aacaaagttt tttgga |

By “genetic alteration in a Jarid2 nucleic acid molecule” is meant any alteration in the sequence of a Jarid2 nucleic acid molecule relative to a reference sequence. Jarid2 reference sequences include any wild-type Jarid2 nucleic acid molecule provided herein. Exemplary genetic alterations include, but are not limited to, any one or more of the following polymorphisms: rs7766973, rs6915344, rs12530202, rs2295954, rs9464779, rs11962776, rs6921502, rs9396578, rs6459404, rs9370809, rs3759, rs957387, rs707833, rs13193457, rs909626.

By “JARID2 protein” is meant a polypeptide or a fragment thereof having at least 85% amino acid sequence identity to NCBI Accession No. NP_{—}004964.2 that functions in neurodevelopment and/or embryogenesis.

By “rs7766973” is meant the single nucleotide polymorphism (SNP) located on chromosome 6 that corresponds to the sequence CCCAGAGGGTTTATATTTTACCTGCA[C/T]TCCTGAGGATGTGTTTGTGTT GCTT where the polymorphism can be either a C or a T in the position indicated by brackets.

By “rs6915344” is meant the single nucleotide polymorphism (SNP) located on chromosome 6 that corresponds to the sequence ATGTTCTTTCAATGGAAGCCCCCACC[C/T]TCTGAGTACACTGGTTCATA GTTAT where the polymorphism can be either a C or a T in the position indicated by brackets.

By “rs12530202” is meant the single nucleotide polymorphism (SNP) located on chromosome 6 that corresponds to the sequence GCGAATGCATTTGTTGGGATTGACTT[C/T]AATAATGAGGCTGGTTTTGT TTAAA where the polymorphism can be either a C or a T in the position indicated by brackets.

By “rs2295954” is meant the single nucleotide polymorphism (SNP) located on chromosome 6 that corresponds to the sequence TGTCCTTGAGAAACTCATAAGTTGCA[A/G]TGTAATCCTGTCTTAATTGT GTTGA where the polymorphism can be either an A or a G in the position indicated by brackets.

By “rs9464779” is meant the single nucleotide polymorphism (SNP) located on chromosome 6 that corresponds to the sequence AAAGAGGAACCCTACTGGTAGAAGTT[C/T]TTGAGAGCTATTCTTGAGAG CTGGT where the polymorphism can be either a C or a T in the position indicated by brackets.

By “rs11962776” is meant the single nucleotide polymorphism (SNP) located on chromosome 6 that corresponds to the sequence TGCAAAAAAGGGACAGTCAGATTAAA[A/C]TGTGGACAGCAGAGTAGTT GTTCAT where the polymorphism can be either an A or a C in the position indicated by brackets.

By “rs6921502” is meant the single nucleotide polymorphism (SNP) located on chromosome 6 that corresponds to the sequence AACCTCTGTTTTGTTGGGTTACCTCC[A/G]TCTCTGTGACTTGGGGTGACA ACCT where the polymorphism can be either an A or a G in the position indicated by brackets.

By “rs9396578” is meant the single nucleotide polymorphism (SNP) located on chromosome 6 that corresponds to the sequence CCCTTCCCTCACTGACTTTATATTTC[G/T]GGAAATTTCATGTCTAGGGAA GTTG where the polymorphism can be either a G or a T in the position indicated by brackets.

By “rs6459404” is meant the single nucleotide polymorphism (SNP) located on chromosome 6 that corresponds to the sequence GAGATGCAGCTTCCAGTCAGTGCGCA[C/T]ATACCACTTGGAGGGCATGC TGGTT where the polymorphism can be either a C or a T in the position indicated by brackets.

By “rs9370809” is meant the single nucleotide polymorphism (SNP) located on chromosome 6 that corresponds to the sequence TTGGAGGGCATGCTGGTTGCAACCCT[C/T]TTATTCTAATAAGGAACTGG TTTGG where the polymorphism can be either a C or a T in the position indicated by brackets.

By “rs3759” is meant the single nucleotide polymorphism (SNP) located on chromosome 6 that corresponds to the sequence AGGATGAGGTGAGCTTACCAACCCCA[A/C]TGAGTAGGGGCCAAACATC CTTAAC where the polymorphism can be either an A or a C in the position indicated by brackets.

By “rs957387” is meant the single nucleotide polymorphism (SNP) located on chromosome 6 that corresponds to the sequence AACATGATTTCCTTCAGCTTCTCCTC[A/G]TATTTACAAGCCAATTGCTTG ACTC where the polymorphism can be either an A or a G in the position indicated by brackets.

By “rs707833” is meant the single nucleotide polymorphism (SNP) located on chromosome 6 that corresponds to the sequence AAACATCTCAAAACTGCACAGAAGAA[C/T]CCATCAAAAAATTTTATGT AACAGT where the polymorphism can be either a C or a T in the position indicated by brackets.

By “rs13193457” is meant the single nucleotide polymorphism (SNP) located on chromosome 6 that corresponds to the sequence CTTACGTTTTCTTAGAGTTACATGGG[A/C]AACATTGTAGTTCAGCACAG CCCTT where the polymorphism can be either an A or a C in the position indicated by brackets.

By “rs909626” is meant the single nucleotide polymorphism (SNP) located on chromosome 6 that corresponds to the sequence AATCCAAATGCCCGTGCAGGAACCCC[A/G]CTCCCCCTGGGATCCTAACA TGGGG where the polymorphism can be either an A or a G in the position indicated by brackets.

By “genetic predisposition” is meant an increased susceptibility to a particular disease due to the presence of one or more genetic alterations relative to a reference sequence.

By “genetic material” is meant nucleic acid molecules from a biological sample obtained from a subject. Genetic material comprises, for example, deoxyribonucleic acid (DNA) or ribonucleic acid (RNA).

By “developmentally disabled child” is meant a child that displays at least one symptom of an autism spectrum disorder.

By “relative” is meant an individual having a genetic relationship with the subject. Exemplary relatives include, but are not limited to grandparents, great-grandparents, siblings, first, second and extended cousins, nieces, nephews, aunts, uncles, parents of a subject.

By “biological sample” is meant any tissue, fluid, or solid material derived from a subject. The sample may be obtained invasively or non-invasively. Preferred biological samples include, blood, feces, urine, semen, mouth swabs, skin cells, nail clippings, hair, amniotic fluid, or cervical smear samples.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of an analyte to be detected. In one embodiment, “detect” refers to identifying the presence or absence of a genetic alteration in genetic material derived from a subject.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “hybridize” is meant pair to form a double-stranded molecule between complementary polynucleotide sequences (e.g., genes listed in Tables 1 and 2), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in sequence, expression level or activity that is associated with a disease or disorder.

By “genetic alteration” is meant any alteration in a nucleic acid sequence relative to a reference. Desirably, the nucleic acid sequence has at least one base pair alteration from a reference sequence. Genetic alterations include, but are not limited to, substitutions, insertions, deletions, or frameshift mutations

As used herein, “obtaining” as in “obtaining an agent” includes synthesizing, purchasing, or otherwise acquiring the agent.

By “polymorphism” is meant a sequence alteration present in 1% or more of alleles of the general population. A polymorphism present both in patients having a disease (e.g., autism or autism spectrum disorder) and in the general population is not necessarily benign. A polymorphism may increase the risk that a subject has or will develop a disease. A polymorphism predisposes a subject to a disease when the polymorphism shows a statistically significant association with the disease.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰° indicating a closely related sequence.

By “subject” is meant a mammal, including, but not limited to, a human or non-human mammal, such as a bovine, equine, canine, ovine, or feline. Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 shows the genome-wide association results. FIG. 1 is a graphical presentation of the TDT results. Each chromosome is represented by a different color. The blue line indicates P<10⁻⁴, the significance at which attempts were made to replicate results, and the red line indicates P<10⁻⁵. Data include those passing a final quality control.

FIG. 2 shows the results of the association test for 48 SNPs located in in the JARID2 gene. The −log₁₀ of the P value of the test is shown for the different SNPs ordered by their position. The solid black line represents the log transformation of the α_{N}=5% threshold P value and the red line represents the log transformation of the corrected 5% threshold (α_{C}=0.0002962) P value.

FIG. 3 shows the linkage disequilibrium for SNPs associated at the nominal level in the JARID2 gene. Linkage disequilibrium is defined using the D′ coefficient using Haploview software (Barrett et al. 2005). Framed SNPs indicate markers associated at the nominal level. The red arrow indicates the position of SNP rs7766973 from Weiss et al (Weiss et al. 2009).

### DETAILED DESCRIPTION OF THE INVENTION

The invention features compositions and methods that are useful for the diagnosis, treatment, and prevention of autism.

The invention is based, at least in part, on the discovery that polymorphisms in the JARID2 gene are genetically linked with autism. In particular, a polymorphism in the rs7766973 SNP is associated with autism. Accordingly, the invention provides diagnostic compositions that are useful in identifying subjects as having or having a propensity to develop an autism spectrum disorder, as well as methods of using these compositions to determine a subject's prognosis or selecting a treatment regimen.

### Autism and Autism Spectrum Disorders

Autism and autistic spectrum disorders are characterized by a variety of behavioral, clinical, and biochemical abnormalities. Autism and autistic spectrum disorders have been shown to have a genetic basis. In particular, family studies have determined that there is a 75-fold greater chance of siblings inheriting autism or an autistic spectrum disorder than the general population if a brother or sister already has autism (Bolton, et al. (1994) J. Child Psychol. Psychiat. 35:877-900). Monozygotic twin studies display a 75% concordance in symptoms in comparison to only 10% in fraternal twins (Bailey, et al. (1998) Brain 121:889-905; Folstein and Rutter (1977) J. Child Psychol. Psychiat. 18:297-321; Ritvo, et al. (1985) Am. J. Psychiat. 142:74-77). These large differences indicate that multiple loci are involved in autism, with some models predicting more than 15 genes contributing to the disorder (Folstein and Rosen-Sheidley (2001) Nat. Rev. Genet. 2:943-955; Lamb, et al. (2000) Hum. Mol. Genet. 9:861-868; Risch, et al. (1999) Am. J. Hum. Genet. 65:493-507).

The characteristic behaviors of autism and autism spectrum disorders may be apparent in infancy (18 to 24 months), but usually do not become obvious until early childhood (24 months to 6 years). In general, children having or suspected of having autism or an autistic spectrum disorder display delays or disruptions in social and communication. Research indicates that early diagnosis of autism or autism related disorders (e.g., Asperger's pervasive developmental disorder) may be associated with dramatically better outcomes. The earlier a child is diagnosed, the earlier the child can benefit from therapeutic intervention.

### Diagnostic Assays

The present invention features compositions and methods useful in identifying a subject as having or having a genetic predisposition to develop autism or an autism spectrum disorder. In general, the methods involve detecting a genetic alteration in a JARID2 nucleic acid sequence isolated from a subject or in a genetic sample. Such alterations may be in a coding sequence or in a regulatory sequence that modulates the expression (e.g., the temporal, spatial or level) of a JARID2 polypeptide or polynucleotide. In addition or alternatively, the genetic alteration is detected at a gene map loci that is any one or more of 6p23 (Jarid2), where the genetic map loci shows a statistically significant association with autism or an autism spectrum disorder.

In one embodiment, a subject is identified as at risk of developing autism or an autism spectrum disorder prior to detecting the genetic alteration in JARID2. Subjects at increased risk include those that have a sibling or other relative with autism or an autism spectrum disorder; subjects having a relative diagnosed as having schizophrenia, or subjects that display developmental delays or abnormalities (e.g., speech and communication delays, social delays).

In certain embodiments, polymorphisms in the rs7766973 SNP are measured in a subject sample. In other embodiments, polymorphisms in the JARID2 genes are measured in a subject sample. In other embodiments polymorphisms in at least one of the rs6915344, rs12530202, rs2295954, rs9464779, rs11962776, rs6921502, rs9396578, rs6459404, rs9370809, rs3759, rs957387, rs707833, rs13193457, and rs909626 SNPs are measured in a subject. Standard methods may be used to measure polymorphisms.

Biological samples include tissue samples (e.g., cell samples (e.g., cheek swabs), biopsy samples) and bodily fluids, including, but not limited to, blood, saliva, tears, urine, seminal fluids, and ejaculate. Biological samples may be isolated from the subject or from relatives of the subject, including but not limited to, parents, siblings, grand-parents, uncles, aunts, nieces, nephews, and cousins. In one embodiment, the presence of a particular polymorphism in the rs7766973 SNP indicates the presence of or propensity to develop autism or an autism spectrum disorder, or symptoms thereof. In other embodiments polymorphisms in at least one of the rs6915344, rs12530202, rs2295954, rs9464779, rs11962776, rs6921502, rs9396578, rs6459404, rs9370809, rs3759, rs957387, rs707833, rs13193457, and rs909626 SNPs indicates the presence of autism or an autism spectrum disorder, or a propensity to develop autism or an autism spectrum disorder in a subject.

Any suitable method can be used to detect one or more polymorphisms in JARID2 or at a gene map loci of 6p23 (Jarid2). Successful practice of the invention can be achieved with one or a combination of methods that can detect a genetic polymorphism. These methods include, without limitation, direct sequencing-based methods, hybridization-based methods, primer extension-based methods, ligation-based methods, methods based on the conformation of a molecule containing the polymorphism, and invasive cleavage-based methods. The various methods may be carried out in various reaction formats including homogeneous reactions and reactions on solid supports. Various detection methodologies may be employed in detecting genetic polymorphisms of the claimed invention including, but not limited to, radioactive detection, luminescence detection, fluorescence detection, time-resolved fluorescence detection, fluorescence resonance energy transfer, fluorescence polarization, gel electrophoresis, and mass spectrometry.

Polymorphisms can be detected by direct sequencing. In this approach, genomic regions of interest are amplified by polymerase chain reaction. The amplified PCR products are directly sequenced to determine which of the polymorphic alleles are present in the biological sample obtained from the subject.

Hybridization based genotyping methods include genotyping on a microarray. Under this approach, the genomic regions of interest (those containing the polymorphic alleles associated with an autism spectrum disorder) are amplified by polymerase chain reaction and the reaction products are analyzed by hybridization to target sequences immobilized on a solid support. The solid supports may include commercially available microarrays (e.g. the GeneChip HuSNP Mapping Array). The PCR products are hybridized to the microarray, stained, and visualized. Hybridization to the various allele specific probes is measured and used to determine which alleles are present in the subject. An alternative approach based on hybridization to a solid support is the dynamic allele-specific hybridization assay (DASH). In this approach, instead of measuring specific hybridization under constant conditions, the melting temperature differences between the various PCR products annealed to allele-specific probes are determined. Base pair differences between the different alleles result in mismatches in the annealed product. The mismatches result in lower melting temperatures compared to DNA duplexes without mismatches. The specific alleles present in a subject can thus be determined from the melting point of the subject specific PCR product.

Polymorphic alleles can also be detected using molecular beacons, which are nucleic acid molecules having a fluorescent molecule on one end and a quencher molecule on the other end. Molecular beacons form a stem-loop structure such that the fluorescent molecule is positioned close to the quencher. Fluorescence is only observed when the stem-loop structure unfolds—for example, when the molecular beacon hybridizes to a complementary sequence. Molecular beacons can be designed such that they only hybridize (and thus fluoresce) to a nucleic acid molecule corresponding to a specific allele. Genomic regions of interest may be amplified from a subject and denatured. The denatured PCR products are then mixed with the appropriate molecular beacons. The various alleles carried by the subject are then determined by measuring the fluorescent signal obtained from those molecular beacons that hybridize to their specific target allele.

The 5′-nuclease activity of Taq DNA polymerase can also be used to determine which allele is present in a particular sample. The 5′-nuclease activity displaces and cleaves an oligonucleotide hybridized to a DNA template undergoing Taq DNA polymerase mediated replication. Assays for specific alleles can be developed using oligonucleotides that contain a fluorescent reporter and a quencher. These oligonucleotides are designed so that they will only hybridize to specific alleles. PCR amplification in the presence of these oligonucleotides generate a fluorescent signal only in the presence of the specific allele in the subject DNA. Polymorphisms may also be detected using allele-specific PCR primers. In this case, amplification primers are designed that are specific for a given allele. Accordingly, the presence of a PCR product is indicative of a particular allele in the subject.

Primer extension-based methods may be used to determine the presence of a particular allele. The regions of interest are amplified from a sample obtained from a subject. A nucleic acid probe is designed to hybridize immediately adjacent to the specific polymorphic allele (for example adjacent to a SNP). DNA polymerase is used to extend the reaction which will incorporate the base corresponding to the specific allele into the primer. The reaction can be carried out in the presence of fluorescently labeled dideoxynucleotides (ddNTP) or fluorescently labeled deoxynucleotides (dNTP). In the presence of ddNTPs, a single labeled nucleotide is incorporated into the probe. Each ddNTP may be labeled with a different fluorescent probe, thus allowing the determination of the specific alleles present in a sample. In the presence of dNTPs, the primer extension product is further hybridized to a set of labeled nucleic acids that each correspond to a particular allele. The specific alleles present in the subject are then determined based on hybridization. Alternatively, mass spectrometry can be used to detect the presence of various primer extension products. In this case, extension is carried out in the presence of ddNTPs where the various products corresponding to different alleles can be distinguished by molecular mass. Furthermore, primer extension can be carried out in the presence of hapten-labelled nucleotides. The haptens incorporated into the primer extension products are then detected by using antibodies specific for each hapten.

The presence of various alleles can be determined through the use of ligase assays. DNA ligase catalyzes the ligation of the ‘3 end of a DNA molecule to the 5’ end of a DNA molecule directly adjacent to the first DNA molecule. A set of DNA probes can be designed so that various combinations of DNA molecules will be adjacent depending on the specific allele present in the sample. For example, four distinct probes can be generated, each differing at the 5′-nucleotide. Only the probe containing the 5′ nucleotide present in the specific allele will be ligated to the adjacent 3′ nucleotide. The ligated products are then measured to determine which alleles are present in the sample.

Various alleles can also be detected by using invader assays. The invader assay relies on the activity of flap endonuclease, an endonuclease that cleaves at specific nucleic acid structures. Two probes are generated. The first probed, termed the invader probe, is an oligonucleotide that is complementary to the 3′ end of the target DNA obtained from the subject. The second probe is an allele-specific probe which is complementary to the 5′-end of the of the target DNA, but also extends past the 3′ side of the SNP allele. The allele-specific probe contains a base that is complementary to a specific SNP allele. If the target DNA contains the particular allele, the invader probe and the allele-specific probe will both bind to the target DNA and form a structure that is recognized and cleaved by flap endonuclease. If the target does not contain the specific allele, the probes do not form the appropriate structure and flap endonuclease does not cleave the product. The probes can be designed such that flap endonculease cleavage generates a signal that can be detected. For example the allele-specific probe can have a fluorescent probe on one end and a quencher on the other. Flap endonuclease cleavage liberate the fluorescent moiety away from the quencher and produces a fluorescent signal.

Polymorphisms can also be detected through the use of methods that rely on differences in the conformation, weight, or size of molecules carrying various polymorphisms. An example of such an approach is single-strand conformation polymorphism (SSCP). The mobility of single stranded DNA in gel electrophoresis is affected by very small changes in the sequence of the DNA. These small changes result in noticeable mobility shifts because of the relatively unstable nature of single stranded DNA. These conformational changes may be detected by gel electrophoresis or chromatography. Preferably the conformational changes can be detected using denaturing high performance liquid chromatography.

### Autism Clinical Criteria

While there is no one behavioral or communications test that can detect autism, several screening instruments have been developed that are now used in diagnosing autism. CARS rating system (Childhood Autism Rating Scale), developed in the early 1970s, is based on observed behavior. Using a 15-point scale, professionals evaluate a child's relationship to people, body use, adaptation to change, listening response, and verbal communication. The Checklist for Autism in Toddlers (CHAT) is used to screen for autism at 18 months of age. It was developed in the early 1990s to see if autism could be detected in children as young as 18 months. The screening tool uses a short questionnaire with two sections, one prepared by the parents, the other by the child's family doctor or pediatrician. The Autism Screening Questionnaire is a 40-item screening scale that has been used with children four and older to help evaluate communication skills and social functioning. The Screening Test for Autism in Two-Year Olds uses direct observations to study behavioral features in children under two. Three skills areas have been identified—play, motor imitation, and joint attention—that seem to indicate autism. The diagnostic criteria for Asperger's Disorder include 1) a qualitative impairment in social interaction, as manifested by at least two of the following: marked impairments in the use of multiple nonverbal behaviors such as eye-to-eye gaze, facial expression, body postures, and gestures to regulate social interaction; failure to develop peer relationships appropriate to developmental level; a lack of spontaneous seeking to share enjoyment, interests, or achievements with other people (e.g., by a lack of showing, bringing, or pointing out objects of interest to other people); or lack of social or emotional reciprocity, and 2) restricted repetitive and stereotyped patterns of behavior, interests, and activities, as manifested by at least one of the following: encompassing preoccupation with one or more stereotyped and restricted patterns of interest that is abnormal either in intensity or focus; apparently inflexible adherence to specific, nonfunctional routines or rituals; stereotyped and repetitive motor mannerisms (e.g., hand or finger flapping or twisting, or complex whole-body movements); or persistent preoccupation with parts of objects. Asperger's Disorder causes clinically significant impairment in social, occupational, or other important areas of functioning. There is no clinically significant general delay in language (e.g., single words used by age 2 years, communicative phrases used by age 3 years). There is no clinically significant delay in cognitive development or in the development of age-appropriate self-help skills, adaptive behavior (other than social interaction), and curiosity about the environment in childhood. Criteria are not met for another specific Pervasive Developmental Disorder or Schizophrenia.

Because a behavior-based diagnosis of autism is difficult, especially in young patients, it is of value to have a genetic screening assay to assist in the diagnosis. Furthermore, such an assay can be used to advise potential parents of their chances of having autistic children. However, up until the disclosure in the present invention, there has been no genetic marker available which is indicative of a subject's susceptibility to autism, or a disease related thereto, such as Asperger's Disorder or PDD-NOS.

As used in the context of the present invention, autism includes any form of autism. Such diseases are currently denoted as autism or an autistic spectrum disorder which includes Asperger's Syndrome and Pervasive Developmental Disorder (PDD-NOS).

The “DSM-IV” criteria for autistic disorder are those set forth in the American Psychiatric Association's Diagnostic and Statistical Manual of Mental Disorders, 4.sup.th edition, Washington, D.C., 199 pp 70-71. The diagnosis is based on the presence of 6 or more diagnostic criteria from three possible Groups 1, 2, and 3 with at least two of the criteria being from Group 1, at least one from Group 2, and at least one from Group 3. The three groups correspond to three core symptoms:

Group 1—Qualitative impairment in social interaction.

Group 2—Qualitative impairment in communication.

Group 3—Restricted repetitive and stereotyped patterns of behavior, interests and activities.

The four diagnostic criteria in Group 1 are: (i) marked impairment in multiple nonverbal behaviors (e.g., eye to eye gaze, facial expression, body postures, and gestures to regulate social interaction); (ii) failure to develop peer relationships appropriate to developmental level; (iii) absence of spontaneous seeking to share enjoyment, interests, or achievements with others (e.g., lack of showing, bringing, or pointing out objects of interest); and (iv) absence of social or emotional reciprocity.

The four diagnostic criteria in Group 2 are: (i) delay in, or total absence of spoken language development (without an attempt to compensate through alternative modes of communication, such as gesture or mime); (ii) adequate speech but marked impairment in ability to initiate or sustain a conversation with others; (c) stereotyped and repetitive use of language or idiosyncratic language; and (iv) absence of varied, spontaneous make-believe play or social imitative play appropriate to developmental level.

The four diagnostic criteria in Group 3 are: (i) encompassing preoccupation with one or more stereotyped and restricted patterns of interest that is abnormal either in intensity or focus; (ii) inflexible adherence to specific nonfunctional routines or rituals, stereotyped and repetitive motor mannerisms (e.g., hand or finger flapping or twisting, or complex whole-body movements); and (iv) persistent preoccupation with parts of objects.

Diagnosis of autism using the DSM-IV criteria, followed by confirmation of the diagnosis by the Autism Diagnostic Interview-Revised (ADI-R), provides good results. The ADI-R is a well-known review process for establishing autism (see, for example, Lord, et al. (1994) J. Autism Dev. Disord. 2:659-85; Le Couteur, et al. (1989) J. Autism Dev. Disord. 19:363-87).

Accordingly, clinical criteria diagnostic of autism may be used to select subjects who could benefit from genetic testing for genetic alterations in JARID2 nucleic acid molecules. If desired, results of genetic testing for genetic alterations in JARID2 nucleic acid molecules are used alone or in combination with diagnostic criteria to identify subjects as having or having a propensity to develop autism or an autism spectrum disorder.

### Diagnostic Kits

The invention provides kits for diagnosing autism or an autism spectrum disorder or the propensity to develop such disorders, or for identifying a subject as in need of therapeutic intervention for the treatment of autism or an autism spectrum disorder. In one embodiment, the kit includes a composition containing at least one agent that is useful in detecting a genetic polymorphism associated with autism. In another embodiment, the kit includes a probe nucleic acid that binds to a site adjacent to a polymorphism associated with autism such that the probe can be used as a primer for direct sequencing or a primer extension assay. In other embodiments, the kit includes a molecular beacon that can be used in an assay to detect a polymorphism associated with autism. In further embodiments, the kit includes a microarray that can be used to detect a polymorphism associated with autism. In yet another embodiment, the kit includes a composition containing at least one agent that binds a polypeptide or polynucleotide whose expression is increased in autism. In another embodiment, the invention provides a kit that contains an agent that binds a nucleic acid molecule whose expression is altered in autism. In some embodiments, the kit comprises a sterile container which contains the binding agent; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired the kit is provided together with instructions for using the kit to diagnose autism. The instructions will generally include information about the use of the composition for diagnosing a subject as having autism or having a propensity to develop autism. In other embodiments, the instructions include at least one of the following: description of the binding agent; warnings; indications; counter-indications; animal study data; clinical study data; and/or references. The instructions may be printed directly on the container (when present), or as a label applied to the container, or as a separate sheet, pamphlet, card, or folder supplied in or with the container.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Genome Wide Association Analysis in Autism

For a high-resolution genetic study of autism, families were selected that included multiple affected individuals (multiplex) from the widely studied Autism Genetic Resource Exchange (AGRE) and US National Institute for Mental Health (NIMH) repositories Although the phenotypic heterogeneity in autism spectrum disorders is extensive, in the primary screen families were selected in which at least one proband met ADIR criteria for diagnosis of autism and included additional siblings in the same nuclear family

The families and samples from two sources were combined for the genetic association screen. The Autism Genetic Resource Exchange (AGRE) sample included nearly 3,000 individuals from over 780 multiplex autism families in the AGRE collection genotyped at the Broad Institute on the Affymetrix 5.0 platform, which includes over 500,000 single-nucleotide polymorphisms (“SNPs”). The NIMH sample included a total of 1,233 individuals from 341 multiplex nuclear families (258 of which were independent of the AGRE sample) genotyped at the Johns Hopkins Center for Complex Disease Genomics on Affymetrix 5.0 and 500K platforms, including the same SNP markers as were genotyped in the AGRE sample.

Before merging, each data set was carefully filtered separately to ensure the highest possible genotype quality for analysis, since technical genotyping artifacts can create false positive findings. The distribution of χ² values for the highest quality data was examined, and a series of quality control (QC) filters designed to identify a robust set of SNPs were used, including data completeness for each SNP, Mendelian errors per SNP and per family, and a careful evaluation of inflation of association statistics as a function of allele frequency and missing data. As 324 individuals were genotyped at both centers, a concordance check was performed to validate this approach. After excluding one sample mix-up, an overall genotype concordance between the two centers of 99.7% for samples typed on 500K at JHU and 5.0 at Broad and 99.9% for samples run on 5.0 arrays at both sites was obtained. The combined dataset, consisting of 1,031 nuclear families (856 with two parents) and a total of 1,553 affected offspring, was employed for genetic analyses. These data were publicly released in October, 2007 and are directly available from AGRE and NIMH.

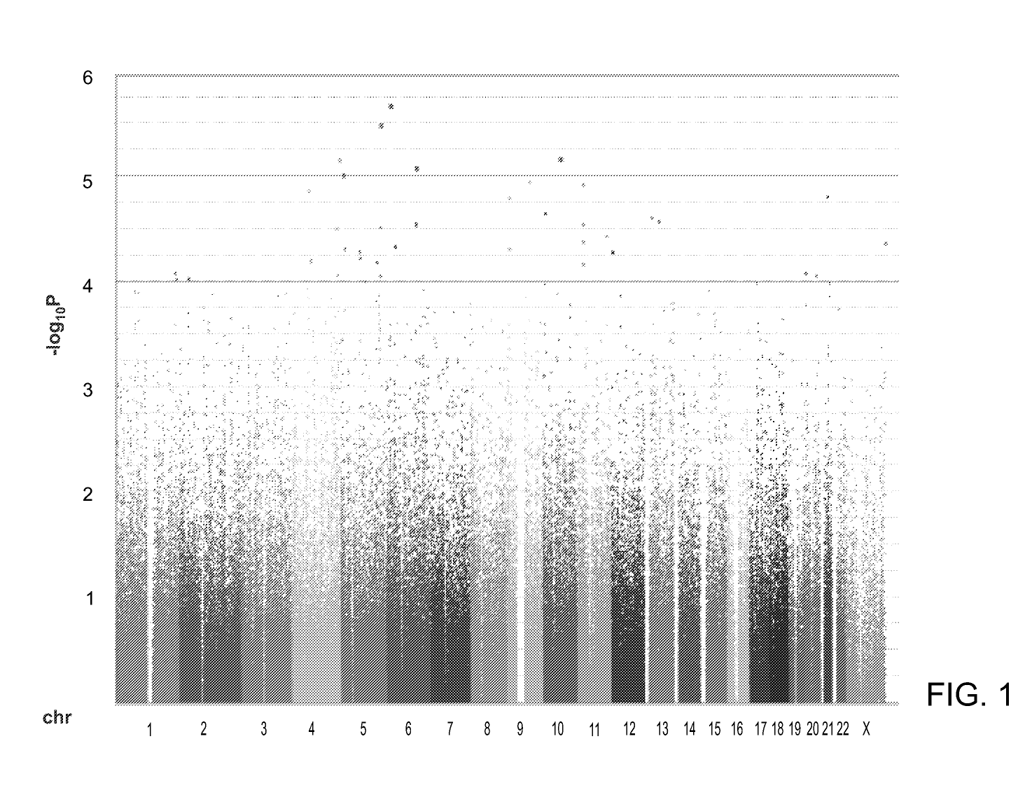

The transmission disequilibrium test (TDT) across all SNPs passing quality control in the complete family dataset was used for association analyses since the TDT is not biased by population stratification. Association analysis was performed in PLINK (Purcell et al. 2007). A threshold for genome-wide significance was estimated using both permutation (P<2.5×10⁻⁷) and estimating the effective number of tests (P<3.4×10⁻⁷) and use the more conservative. No SNP met criteria for genome-wide significance at P<2.5×10⁻⁷. However, an excess of independent regions associated at P<10⁻⁵ (6 observed vs. 1 expected) and P<10⁻⁴ (30 observed vs. 15 expected) were observed despite the lack of overall statistical inflation ( ), indicating that common variants in autism exist, but that the initial scan did not have sufficient statistical power to identify them definitively (FIG. 1).

The strongest association was found at chromosome 6p23 (rs7766973, P=6.8×10⁻⁷) in JARID2, an ortholog of the mouse jumonji gene, encoding a nuclear protein that functions in embryogenesis, especially neural tube formation. During the scan 631 counts were transmitted and 811 counts were un-transmitted yielding an odds ratio of 0.78 (P value 6.8×10⁻⁷) for the minor allele of rs7766973.

### Example 2

### Analysis of Schizophrenia Associated Genes in Autism Families

Since schizophrenia and autism are two psychiatric diseases known to share a common genetic background (Crespi et al. 2009), five genes clearly implicated in schizophrenia (DTNBP1, NRG1, DAOA, DISC1 and JARID2) were evaluated for their role in autism using a family-based association analysis conducted using 856 nuclear families with autistic children and 441 SNPs covering the five genes that were available from a previous GWAS study.

Nine hundred and eight families were available from the AGRE collection (Geschwind et al. 2001) genotyped with Illumina 550K at the Children's Hospital of Philadelphia (CHOP). This sample including 1816 founders, 1519 children with an autism diagnosis (1219 males and 300 females; male:female sex ratio=4.06) according to the Autism Diagnostic Interview Revisited (ADI-R). Use of homogeneous definition of autism (compared to the broader autism spectrum disorder phenotype definition) is one way to improve the power in complex disease study. Exclusion criteria for children included a diagnosis of Rett syndrome and childhood disintegrative disorder, as defined by the Diagnostic and Statistical Manual of Mental Disorders, fourth edition (DSM-IV) criteria for other pervasive developmental disorders, presence of a known genetic condition, history of serious head injury or neurologic disease, or significant sensory or motor impairment.

Genotype data were checked for Mendelian inconsistency per SNP and per family, and missing genotype frequency per SNP and per individuals using PEDSTATS (Wigginton and Abecasis, 2005). SNPs with a minor allele frequency (MAF) lower than 5% were excluded and Hardy-Weinberg equilibrium (HWE) was checked in founders using the exact test (Wigginton et al. 2005). Haploview (Barret et al. 2005) was used to estimate linkage disequilibrium coefficient, D′ and r2, and provide a graphical representation.

3190 individuals remained from 856 families, including 1712 founders and 1479 affected children (1189 males and 289 females; male:female sex-ratio=4.11). From the 485 SNPs available in the Illumina 550K array that cover the 5 genes, 441 passed the QC among which 91 were located within the JARID2 gene region.

SNPs association analyses were conducted using the Pedigree Disequilibrium Test (PDT) (Martin et al. 2000) method as implemented in UNPHASED (Dudbridge 2003, 2008) version 3.0.12 under an additive transmission model and p-values were estimated based on 1,000 permutations.

To account for multiple testing, a correction of the nominal P value (5%) is applied dividing this value by the number of test (Bonferroni correction). This correction assumes independence of the tests. In the case of SNPs in linkage disequilibrium, the independence assumption is violated and the Bonferroni correction becomes too conservative. Others have proposed a method to estimate the effective number of independent tests based on a spectral decomposition of the correlation matrix for the SNP (MatSpD) (Li and Ji, 2005). This methodology takes into consideration linkage disequilibrium patterns among all SNPs and reduces this set to a minimal number of “effective” SNPs. In the present data, the set of 441 SNPs was reduced to 217.7553 SNPs that transformed the 0.05 nominal P value to a 0.00023 corrected significativity threshold. A Sidak correction (Sidak 1968, 1971), less conservative than the Bonferroni correction, was then applied to the P value obtained from UNPHASED to obtain a corrected P value (P_{corrected}) for each marker.

In genes with significantly associated SNP, conditional analysis was conducted to test whether risk allele at particular SNPs accounted for the association signal in the region. If the association signal in the region was driven by one or more single SNP, conditioning on its/their effect was expected to remove all evidence of association for the remaining SNPs. This analysis was done using the “-condition” option in UNPHASED.

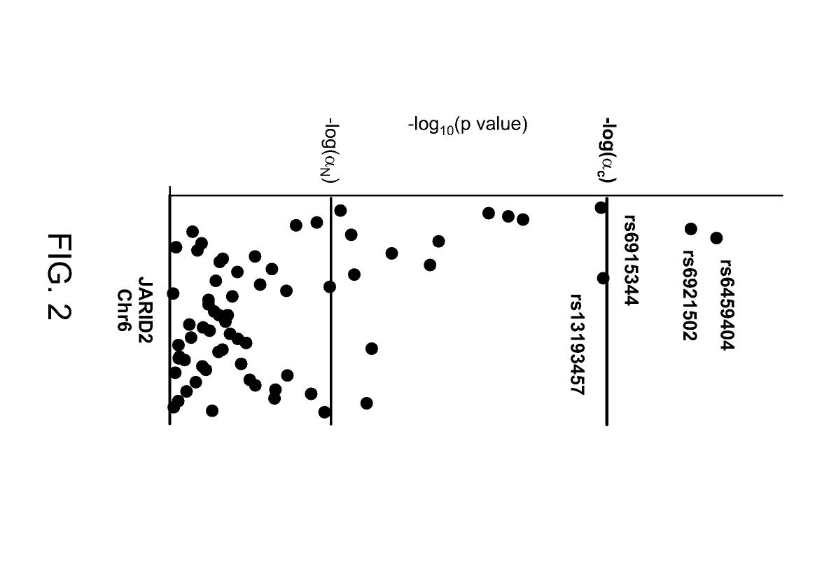

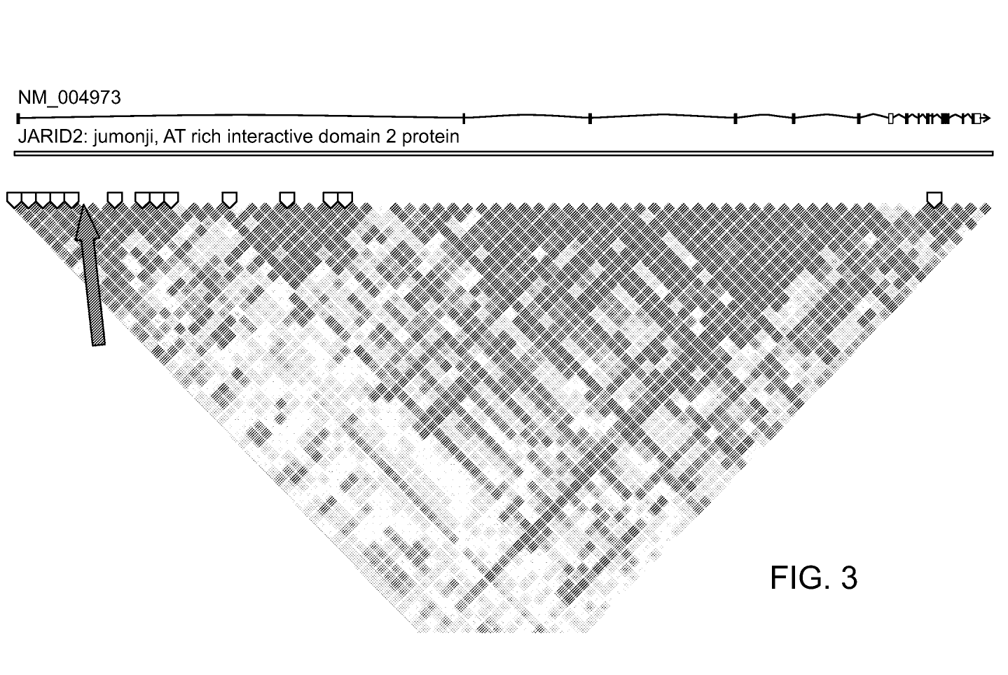

The results of the association tests for the SNPs located in the JARID2 gene are shown in FIG. 2 and individually in Table 1 with a description of their MAF and associated odds ratio (OR). When P values were adjusted for multiple tests, two SNPs remained significant, rs6459404 (OR=1.21, P_{corrected}=0.008) and rs6921502 (OR=0.81, P_{corrected}=0.013), and two other were borderline, rs6915344 (OR=1.21, P_{corrected}=0.069) and rs13193457 (OR=1.43, P_{corrected}=0.067) all in the same linkage disequilibrium (LD) region of JARID2 (FIG. 5). Fourteen SNPs were associated at the nominal level and all except one belonged to the same 5′ region with strong LD (FIG. 3), the same region that contain rs7766973. These SNP with P<0.05 in JARID2 were tested conditioning on rs6459404 the most strongly associated markers in the gene to test if this SNP alone explained the association signal in this gene. SNP rs6921502, the other SNP with rs6459404 that was significantly associated (P_{corrected}<0.05) was not anymore significant at the nominal level (P=1.00). This is explained by their strong LD (D′ and r2 close to one) meaning that they bring the same information. SNP rs13193457 was still associated (P=0.002) as rs12530202, rs2295954, rs11962776 and rs957387 to a lesser extent with p values ranged between 0.01 and 0.05. When these SNPs were tested conditionally on rs6459404 and rs13193457 none except rs957387 remained nominally significant (P=0.0097). These three markers are in strong LD, but could not be distinguished statistically in the present sample. One explanation should be that the causal variant is an untyped variant in strong LD with these markers. Haplotype analysis of the 3 SNPs allowed evaluation of the association of this unobserved deleterious variant. Using UNPHASED, the 3 marker haplotypes displayed a very strong association (stronger than when considering SNPs one at a time) with P=2.5×10⁻⁷. These results replicate the implication of JARID2 in autism and particularly the region of strong LD in the 5′ region of the gene that contain rs7766953 from the previous example.

TABLE 1
SNPs associated at the nominal level with their associated P value (P),
odds ratio (OR and 95% confidence intervals (CI), and corrected P value
(P_{corrected}). In bold, SNPs that have been meta-analyzed
in the SzGene database

| | chro- | | | | | |
|---|---|---|---|---|---|---|
| | mo- | | | | OR** | |
| SNP | some | Gene | MAF* | P | (95% CI) | P_{corrected} |
| rs6915344 | 6 | JARID2 | 0.47 | 0.0003296 | 1.21 | 0.069 |
| | | | | | 1.09-1.35 | |
| rs12530202 | 6 | JARID2 | 0.14 | 0.0424 | 1.17 | 1.000 |
| | | | | | 1.01-1.35 | |
| rs2295954 | 6 | JARID2 | 0.1 | 0.0026520 | 1.32 | 0.439 |
| | | | | | 1.10-1.58 | |
| rs9464779 | 6 | JARID2 | 0.43 | 0.0018460 | 0.85 | 0.331 |
| | | | | | 0.77-0.94 | |
| rs11962776 | 6 | JARID2 | 0.11 | 0.0014080 | 1.32 | 0.264 |
| | | | | | 1.11-1.57 | |
| rs6921502 | 6 | JARID2 | 0.49 | 0.0000622 | 0.81 | 0.013 |
| | | | | | 0.73-0.90 | |
| rs9396578 | 6 | JARID2 | 0.18 | 0.0345 | 1.16 | 1.000 |
| | | | | | 1.01-1.33 | |
| rs6459404 | 6 | JARID2 | 0.49 | 0.0000387 | 0.81 | 0.008 |
| | | | | | 0.73-0.89 | |
| rs9370809 | 6 | JARID2 | 0.33 | 0.0068 | 1.17 | 0.774 |
| | | | | | 1.04-1.31 | |
| rs3759 | 6 | JARID2 | 0.16 | 0.0163 | 1.18 | 0.972 |
| | | | | | 1.03-1.35 | |
| rs957387 | 6 | JARID2 | 0.09 | 0.0079 | 1.28 | 0.821 |
| | | | | | 1.07-1.54 | |
| rs707833 | 6 | JARID2 | 0.37 | 0.0324 | 1.12 | 0.999 |
| | | | | | 1.01-1.25 | |
| rs13193457 | 6 | JARID2 | 0.08 | 0.0003171 | 1.43 | 0.067 |
| | | | | | 1.17-1.74 | |
| rs909626 | 6 | JARID2 | 0.16 | 0.0256 | 0.85 | 0.996 |
| | | | | | 0.73-0.98 | |

*Minor Allele Frequency
**Odds ratio (OR) with the MAF allele as reference

Materials and Methods

The results described above were obtained using the following methods and materials.

### Samples and Genotyping

The primary samples are from the AGRE and NIMH Repositories. Replication with Affymetrix technology included NIMH controls, families collected by members of the Autism Consortium, and families ascertained from Montreal. Replication with Sequenom technology included the Autism Genome Project, Finnish, and Iranian subsets of Autism Consortium investigator-collected families Details of the ascertainment for each sample collection, genotyping, and quality control processes is provided herein.

### Linkage and Association Analysis

The linkage analysis was conducted with a pruned autosomal SNP set and chromosome X set (670 SNPs) using the cluster option in MERLIN/MINX (r²<0.1)(23), yielding 16,581 independent markers. Confirmatory analysis was performed on non-overlapping datasets by selecting alternate SNPs.

Association analysis was performed in PLINK (24). The basic association test was a transmission disequilibrium test (TDT), and the extra cases vs. controls analysis was performed by allelic association, after excluding cases that were not well-matched to the controls, based on multidimensional scaling (λ<1.1). Combining the TDT and case-control tests was performed using expected and observed allele counts by the formula: Zmeta=(Σexp−Σobs)/√Σvar. Meta-analysis of AGRE/NIMH and replication data was performed using the statistic (Z_{AGRE/NIMH}+Z_{replication})/√². Gene-set analysis was performed in PLINK using the set-based TDT. Imputation-based association was performed in PLINK with the proxy-tdt command, using the HapMap CEU parent samples as the reference panel and information score >0.8. Haplotype analysis in the linkage regions was performed using 5-SNP sliding windows, as implemented in PLINK hap-tdt.

### Primary Study Samples

All samples used in this study arose from investigations approved by the individual and respective Institutional Review Boards in the USA and at international sites where relevant. Informed consent was obtained for all adult study participants; for children under age 18, both the consent of the parents or guardians and the assent of the child were obtained.

The Autism Genetic Resource Exchange (AGRE) curates a collection of DNA and phenotypic data from multiplex families with autism spectrum disorder (ASD) available for genetic research. Individuals from 801 families were genotyped, selecting those with at least one child meeting criteria for autism by the Autism Diagnostic Interview-Revised (ADI-R), while the second affected child had an AGRE classification of autism, broad spectrum (patterns of impairment along the spectrum of pervasive developmental disorders, including PDD-NOS and Asperger syndrome) or Not Quite Autism (NQA, individuals who are no more than one point away from meeting autism criteria on any or all of the social, communication, and/or behavior domains and meet criteria for “age of onset”; or, individuals who meet criteria on all domains, but do not meet criteria for the “age of onset”). Probands with widely discrepant classifications of affection status via the ADI-R and ADOS that could not be reconciled were excluded. Families with known chromosomal abnormalities (where karyotyping was available), and those with inconsistencies in genetic data (generating excess Mendelian segregation errors or showing genotyping failure on a test panel of 24 SNPs used to check gender and sample identity with the full array data) were also excluded. The self-reported race/ethnicity of these samples is 69% white, 12% Hispanic/Latino, 10% unknown, 5% mixed, 2.5% each Asian and African American, less than 1% Native Hawaiian/Pacific Islander and American Indian/Native Alaskan.

The NIMH Autism Genetics Initiative maintains a collection of DNA from multiplex and simplex families with ASD. Individuals from 341 nuclear families were genotyped: 258 of which were independent of the AGRE dataset, with at least one child meeting criteria for autism by the ADI-R, and a second child considered affected using the same criteria as described for the AGRE dataset above. Similar exclusion criteria were used, including known chromosomal abnormalities and excess non-Mendelian inheritance. The self-reported race/ethnicity of these samples is 83% white, 4% Hispanic, 2% unknown, 7% mixed, 3% Asian, and 1% African American.

### Primary Study Samples: Merged Data Set for Primary Screening

The Birdseed algorithm was used for genotype calling at both genotyping centers (26,27). As 324 individuals were genotyped at both centers, a concordance check was performed. One sample showed substantial differences between the two centers, but no excess of Mendelian errors, indicating that a sample mix-up occurred in which each center genotyped a different sibling that was identified as the same sample. Excluding this sample, overall genotype concordance between the two centers was 99.72%.

Before merging data, the distribution of chi-square values was examined and a series of quality control (QC) filters designed to identify a robust set of SNPs was used. Filtering AGRE genotypes to 98% completeness and less than 10 MEs was found to be sufficient to remove SNPs that artificially inflated the chi-square distribution for SNPs with MAF (minor allele frequency)>0.05. For MAF<0.05, a much greater inflation (λ=1.17) was observed, due entirely to a strong excess of SNPs with under-transmission of the minor allele (OR<1). While the same filters yielded high-quality results for SNPs with over-transmission of the minor allele (λ=1.04), much stricter filtering was required for rarer SNPs with OR<1 (missing data <0.005). This is not unexpected based on a well-documented bias in the TDT: if missing data are preferentially biased against heterozygotes or rare homozygotes, significant, artificial over-transmission of the common allele is expected (28,29). To achieve comparable quality, the NIMH dataset was filtered on 96% completeness and fewer than 4 MEs. The final QQ plot for the combined dataset is shown in FIG. 5 and has a λ˜1.03. The combined data set, consisting of 1,031 families (856 with two parents) and a total of 1,553 affected offspring, was employed for association testing.

For linkage analyses, the combined AGRE/NIMH dataset was further merged with Illumina 550K genotype data generated at the Children's Hospital of Philadelphia (CHOP) and available from AGRE, adding ˜300 nuclear families (1,499 samples). The extensive overlap of samples between the AGRE/NIMH and the CHOP datasets (2,282 samples) was used to select an extremely high quality set of SNPs for linkage analysis. Specifically, SNPs were required to be on both the Affymetrix 500K/5.0 and Illumina 550K platforms, with >99.5% concordance across platforms. SNPs were further restricted to MAF>0.2, <1% missing data, Hardy Weinberg P>0.01, and no more than 1 ME. This left ˜36,000 SNPs of outstanding quality. For autosomal SNPs, SNPs were further pruned using PLINK to remove SNPs with r²>0.1, yielding 16,311 SNPs.

### Replication Samples

NIMH Control Samples.

Controls obtained from the NIMH Genetics Repository were genotyped on the Affymetrix 500K platform at the Broad Institute Genetic Analysis Platform for another study. Of these, 1,494 matched well with our sample, and were used as controls to compare with the cases and parents in this study.

Montreal Samples.

Subjects diagnosed with autism spectrum disorders with both of their parents were recruited from clinics specializing in the diagnosis of Pervasive Developmental Disorders (PDD), readaptation centers, and specialized schools in the Montreal and Quebec City regions, Canada (31). Subjects with ASD were diagnosed by child psychiatrists and psychologists expert in the evaluation of ASD. Evaluation based on the Diagnostic and Statistical Manual of Mental Disorders (DSM) criteria included the use of the Autism Diagnostic Interview-Revised (ADI-R)(25) and the Autism Diagnostic Observation Schedule (ADOS)(32). As an additional screening tool for the diagnosis of ASD, the Autism Screening Questionnaire, which is derived from the ADI-R, was completed (33). Furthermore, all proband medical charts were reviewed by a child psychiatrist expert in PDD to confirm their diagnosis and exclude subjects with any co-morbid disorders. Exclusion criteria were: (1) an estimated mental age <18 months, (2) a diagnosis of Rett syndrome or Childhood Disintegrative Disorder and (3) evidence of any psychiatric and neurological conditions including: birth anoxia, rubella during pregnancy, fragile X syndrome, encephalitis, phenylketonuria, tuberous sclerosis, Tourette and West syndromes. Subjects with these conditions were excluded based on parental interview and chart review. However, participants with a co-occurring diagnosis of semantic-pragmatic disorder (due to its large overlap with PDD), attention deficit hyperactivity disorder (seen in a large number of patients with ASD during development), and idiopathic epilepsy (related to the core syndrome of ASD) were eligible for the study.

Santangelo EDSP Family Samples.

Families were ascertained for having one or more autistic children and at least one non-autistic child aged 16 or older for an extremely discordant sib-pair linkage study. Recruitment took place in Massachusetts and surrounding states through contacts with parent support and patient advocacy groups, brochures, newsletters, and the study web site. Parents were interviewed about their children, and non-autistic children were interviewed about themselves. An informant/caregiver, usually the proband's mother, was interviewed using the Autism Diagnostic Instrument-Revised (ADI-R) to confirm the diagnosis of autism at age 4-5 years (25,34). Families were included if the affected children met Diagnostic and Statistical Manual of Mental Disorders-IV (DSM-IV) criteria for autistic disorder and their non-autistic siblings (aged 16 and older) did not display any of the broader autism phenotype traits, which were assessed with the (M-PAS-R), the Pragmatic Language Scale (PLS), and the Friendship Interview (35,36). Probands were excluded if they had medical conditions associated with autism such as fragile X syndrome or gross CNS injury, or if they were under four years of age, due to the possible uncertainty in diagnosis at younger ages. Twenty-nine families met eligibility criteria for the study and comprised the final sample for analysis.

High Functioning Autism Family Samples.

Families were included if their affected child had been previously diagnosed with Autism or Asperger syndrome, had a level of intellectual functioning above the range of mental retardation (i.e., Full Scale, Verbal, and Performance IQ>70), chronological age between 6 and 21 years, and an absence of significant medical or neurological disorders (including fragile X syndrome and tuberous sclerosis). Families were ascertained and recruited through the Acute Residential Treatment (ART) programs and outpatient child and adolescent services at McLean Hospital, as well as through associated hospitals and clinics. Brochures and a website were also utilized. Thirty-three families (133 participants) were enrolled in the study. Participation was voluntary.

MGH-Finnish Collaborative Study.

Altogether 58 individuals with a diagnosis of High Functioning Autism (HFA) or Asperger's Syndrome (AS) were recruited in Finland. Fifty-two children and adolescents aged 8 to 15 years were identified from patient-records at the Oulu University Hospital in 2003. These children and adolescents have been evaluated for HFA/AS at the Oulu University Hospital. In addition, six children (3 boys, 3 girls) 11 years of age were recruited from an epidemiological study conducted in 2001(37).

All participants had full scale IQ scores greater than or equal to 80 measured with the Wechsler Intelligence Scale for Children—Third Revision (38). Furthermore, none of the children subjects were diagnosed with other developmental disorders (e.g., dysphasia, fragile X syndrome). Clinical diagnoses of HFA/AS were confirmed by administering the Autism Diagnostic Interview-Revised and the Autism Diagnostic Observation Schedule. Of the 58 participants with HFA/AS, 35 met the diagnostic criteria for AS and 21 met the diagnostic criteria for HFA according to ICD-10 diagnostic criteria (39). Two participants met diagnostic criteria for PDD-NOS; these participants were excluded due to their manifesting different and less severe symptoms than our sample of children with HFA or AS.

Children's Hospital Boston Samples.

Probands with a documented history of clinical diagnosis of ASD were recruited at Children's Hospital Boston. To participate, they had to be over 24 months of age and have at least one biological parent or an affected sibling available. Subjects were excluded if they had an underlying metabolic disorder or any chronic systemic disease, an acquired developmental disability (e.g. birth asphyxia, trauma-related injury, meningitis, etc.), or cerebral palsy. All participants provided informed consent and a phenotyping battery was performed including the Autism Diagnostic Observation Schedule (ADOS), the Autism Diagnostic Interview-Revised (ADI-R) and other measures to assess cognitive status. 75% of subjects with a clinical diagnosis met strict research criteria for ASD on both ADI-R and ADOS. In addition, a complete family and medical history was obtained.

Homozygosity Mapping Collaborative for Autism (HMCA) Samples.

Families with cousin marriages and children affected by autism spectrum disorder (ASD) with or without mental retardation (MR) were recruited by multiple collaborators in the HMCA. The patients from Istanbul were evaluated by a child psychiatrist (Nahit M. Mukaddes) trained in the Autism Diagnostic Observation Schedule (ADOS) and Autism Diagnostic Interview—Revised (ADIR), and who made diagnoses according to DSM-IV-TR criteria and the Childhood Autism Rating Scale (CARS). Patients from Kuwait were enrolled from the Kuwait Centre for Autism by Samira Al-Saad. In Jeddah, Saudi Arabia, patients were evaluated by both a developmental pediatrician (Soher Balkhy) and a pediatric neurologist (Generoso Gascon) and diagnoses were based on DSM-IV-TR criteria. In Lahore, Pakistan, a neurologist (Asif Hashmi) with training in the ADOS and ADI-R diagnosed patients using DSM-IV-TR criteria. In most settings, patients were enrolled from tertiary clinical centers and these patients had standard of care neuromedical assessments, including physical examination, medical and neurological history, fragile X testing, and other genetic and metabolic testing when indicated. MRI was obtained for patients in whom a brain malformation was suspected or seizures were present. In addition, IQ scores (usually from the Stanford-Binet) and adaptive behavior measures were obtained from the patients' existing medical records. Secondary assessments were conducted on the most informative pedigrees by the Boston clinical team in collaboration with local multidisciplinary teams. Clinical members of the Boston team included: developmental psychologists (Janice Ware, Elaine LeClaire, Robert M. Joseph), pediatric neurologists (Ganesh H. Mochida, Anna Poduri), a clinical geneticist (Wen-Han Tan), and a neuropsychiatrist (Eric M. Morrow). The secondary assessment battery was designed to obtain a comprehensive description of current and historical autism symptomatology, cognitive and adaptive functioning, and neurological and physical morphological status in the patient and pedigree. The secondary assessment included: neurologic examination; genetic dysmorphology examination; the CARS; the Social Communication Questionnaire (SCQ) administered with probing on par with the ADI-R by ADI-R reliable examiners; the ADOS (usually Module 1); the Vineland Adaptive Behavior Scales, Second Edition (VAB S-II); Kaufman Brief Intelligence Test, Second Edition (KBIT-II). ADOS assessments were videotaped and dysmorphology findings were photographed for archival purposes.

AGP Samples.

Individuals typically received at least two of three evaluations for autism symptoms: ADI-R, ADOS and clinical evaluation. Of the 1,679 affected individuals from 1,443 families, 966 met criterion for autism on the ADI-R and ADOS and most of these also had a clinical evaluation of autism; 160 affected individuals met criteria for autism on one of the two diagnostic instruments (ADI-R, ADOS) but were missing information on the other instrument; and, 553 individuals met criteria for spectrum disorder on one or both instruments. Affected individuals were recruited from both simplex and multiplex families, 71% of this sample being from multiplex families. The majority of the families were of European ancestry (83%).

Finnish Autism Family Samples.

Families were recruited through university and central hospitals. Detailed clinical and medical examinations were performed by experienced child neurologists as described elsewhere (40). Diagnoses were based on ICD-10(39) and DSM-IV (41) diagnostic nomenclatures. Families with known associated medical conditions or chromosomal abnormalities were excluded from the study. A total of 106 families included 400 individuals for whom genotype data was available. Of these, 111 had a diagnosis of infantile autism and 13 a diagnosis of Asperger syndrome. All families were Finnish, except for one family where the father was Turkish.

Iranian Trio Samples.

Eligible participants in this study were Iranian families with at least one child affected with ASD, including cases of autistic disorder, Asperger syndrome and pervasive developmental disorder-not otherwise specified (PDD-NOS). Eighty families (282 individuals) from Iran were ascertained and assessed. This sample was ascertained by screening and diagnostic testing of over 90,000 preschool children from Tehran in 2004. Diagnoses of children were made according to DSM-IV criteria via the ADI-R and the ADOS. Patients with abnormal karyotypes and dysmorphic features were excluded. Most of the families were fathermother-child trios but some had more than one affected child. All affected biological siblings were assessed with the same diagnostic tools. 80 families (282 individuals) from Iran were obtained and ascertained.

### Affymetrix Genotyping

The AGRE samples were genotyped on Affymetrix 5.0 chips at the Genetic Analysis Platform of the Broad Institute, using standard protocols. The 5.0 chip was designed to genotype nearly 500,000 SNPs across the genome in order to enable genome-wide association studies (26,27). The NIMH controls were genotyped at the Broad Institute using the Affymetrix 500K Sty and Nsp chips, using a similar protocol. The Autism Consortium and Montreal replication samples were also genotyped at the Broad Institute under the same conditions. The NIMH autism samples were genotyped at the Johns Hopkins Center for Complex Disease on the Affymetrix 500K (Nsp and Sty) and 5.0 platforms using similar standard protocols.

Genotype calling for the 5.0 arrays was performed by Birdseed (26,27) and for the 500K arrays was performed by BRLMM. As basic QC filters for the data generated at the Broad Institute, a requirement was set that genotyping was >95% complete for each individual, and that each family had fewer than 10,000 Mendelian inheritance errors across the genome. A requirement was set that each SNP had >95% genotyping, fewer than 15 Mendelian errors, Hardy-Weinberg Equilibrium P>10⁻¹°, and minor allele frequency greater than 1%. For the AGRE sample, this left 2,883 high quality individuals genotyped for 399,147 SNPs with 99.6% average call rate. The basic filters for the data generated at Johns Hopkins were individual call rates >95% for 5.0 arrays and >90% for 500K arrays data, fewer than 5,000 Mendelian errors per family. Only monomorphic SNPs and those with greater than 50% missing data were dropped, for 498,216 SNPs. The combined dataset had nearly 365,000 SNPs passing QC.

### Sequenom Genotyping

SNPs were assayed using Sequenom technology for the AGP samples at three centers, namely Gulbenkian, Mt. Sinai, and Oxford: DNA from 1,629 families representing numerous recruiting sites was genotyped for 54 SNPs. SNPs with >3% missing data, namely rs4690464, rs105 13025, and rs17088296, were excluded from analysis. The next step in the quality control process was to remove families with >4 Mendelian errors, out of 51 remaining loci, under the assumption that this indicated pedigree errors. Data from 110 families were removed due to Mendelian errors. Thereafter, SNPs were removed if they showed excessive Mendelian errors (>16) in the remaining families. Using this criterion, two more SNPs, rs1 55437 and rs1 925058, were removed from analysis. It was apparent that DNA quality varied by study site and could be responsible for concomitant genotype quality differences. Therefore, the rate of missing genotypes per locus and study site was also evaluated. The analyses showed that DNA from a few population samples showed excess missingness for two SNPs, rs4742408 and rs7869239, relative to the remaining population samples. Specifically three population samples showed more than 7% missing genotypes for rs4742408 and rs7869239 whereas the remaining population samples had about 1% or less missing genotypes. Therefore, for these loci the genotypes only from the samples showing excess missingness were deleted. As a final quality control step, missing genotypes for the remaining loci were evaluated. If more than five loci were missing genotypes, the individual's data was removed from analysis. By this criterion 76 additional families became uninformative for family-based association analysis, leaving 1,443 families for association analysis. The Finnish autism samples were genotyped in the Peltonen lab, and the Iranian trios were genotyped at the Broad Institute using very similar protocols. All samples were genotyped using aliquots from the same pooled primers and probes.

### Copy Number Analysis

Because of previous reports of two large (>1 Mb), independent de novo deletions spanning this locus (42), the region surrounding rs10513025 and the entire SEiA5A locus was assessed for copy number variation that could either explain or provide independent evidence of the importance of this region to autism using Birdsuite (26) to analyze all Affymetrix 5.0 samples. Birdsuite genotypes previously annotated common copy number polymorphisms (27) and in parallel searches for novel copy number variants using an HMM. Probe coverage in the region was good, with no 50 kb window having fewer than 10 probes and an average spacing between probes of 2.5 kb, allowing very good sensitivity for CNVs greater than 25 kb. No deletions or duplications were found near this SNP, nor any overlapping the gene SEiA5A. The closest copy number variants upstream and downstream of this SNP appeared to be a rare (˜2-3% frequency, previously annotated CNP) 40 kb deletion from 288 kb from the 3′ end of SEiA5A, and a rare (˜1% frequency, novel) 20 kb deletion 356 kb upstream of the 5′ end of SEiA5A. Each of these appeared to be segregating polymorphisms, but fall far outside of the boundaries of SEiA5A and TAS2R1 and far beyond the linkage disequilibrium block containing rs10513025.

### Determination of Significance

To determine an appropriate experimental threshold for genome-wide significance, permutation was performed on this dataset by gene-dropping, and genome-wide significance was estimated by taking the lowest P-value from each of 1000 permuted datasets and using the 50^{th} as a threshold for P<0.05 experiment-wide significance (P<2.5×10⁻⁷). To calculate an estimate of the effective number of tests (T_{eff}), the following algorithm was used:

1. Start with the most 5′ SNP on a chromosome (SNPij), where i=chromosome, and j=SNP position, and calculate pairwise LD with all downstream SNPs within 1 Mb (r²[SNP_{1,1}×SNP_{1,n}])

2. For SNP_{1,1}, T_{eff(((1,1)}=1−max(r²[SNP_{1,1}×SNP_{1,n}])

3. For chromosome i,

$${T}_{eff ( i )} = {\sum }_{j = 1}^{m} {T}_{eff ( i , j )} ,$$

where m=the total number of SNPs on a chromosome.

$$4. {T}_{eff} = {\sum }_{i = 1}^{23} {T}_{eff ( i )}$$

Since this algorithm only accounts for pair-wise LD, it provides a conservative estimate of the number of effective tests.

### REFERENCES

Abrahams B S, Geschwind D H (2008). Advances in autism genetics: on the threshold of a new neurobiology. Nat Rev Genet 9:341-355. [PubMed: 18414403]
Barrett, J. C., B. Fry, et al. (2005). “Haploview: analysis and visualization of LD and haplotype maps.” Bioinformatics 21(2): 263-5.
Crespi, B., P. Stead, et al. (2009). Evolution in health and medicine Sackler colloquium: Comparative genomics of autism and schizophrenia. Proc Natl Acad Sci USA 107 Suppl 1: 1736-41.
Dudbridge, F. (2003). “Pedigree disequilibrium tests for multilocus haplotypes.” Genet Epidemiol 25(2): 115-21.
Dudbridge, F. (2008). “Likelihood-based association analysis for nuclear families and unrelated subjects with missing genotype data.” Hum Hered 66(2): 87-98.
Geschwind D H, et al (2001). The autism genetic resource exchange: a resource for the study of autism and related neuropsychiatric conditions. Am J Hum Genet 69:463-466
Li, J. and L. Ji (2005). “Adjusting multiple testing in multilocus analyses using the eigenvalues of a correlation matrix.” Heredity 95(3): 221-7.
Martin, E. R., S. A. Monks, et al. (2000). “A test for linkage and association in general pedigrees: the pedigree disequilibrium test.” Am J Hum Genet 67(1): 146-54.
Purcell S, et al (2007). PLINK: a tool set for whole-genome association and population-based linkage analyses. Am J Hum Genet; 81:559-575.
Sidak, Z. (1968). “On multivariate normal probabilities of rectangles.” Ann Math Stat 39: 1425-1434.
Sidak, Z. (1971). “On probabilities of rectangles in multivariate normal Student distributions: Their dependence on correlations.” Ann Math Stat 41: 169-175.
Weiss, L. A., D. E. Arking, et al. (2009). A genome-wide linkage and association scan reveals novel loci for autism. Nature 461(7265): 802-8.
Wigginton, J. E. and G. R. Abecasis (2005). “PEDSTATS: descriptive statistics, graphics and quality assessment for gene mapping data.” Bioinformatics 21(16): 3445-7.
Wigginton, J. E., D. J. Cutler, et al. (2005). “A note on exact tests of Hardy-Weinberg equilibrium.” Am J Hum Genet 76(5): 887-93.
Zafeiriou D I, Ververi A, Vargiami E (2007). Childhood autism and associated comorbidities. Brain Dev 29:257-272.

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A method for determining a genetic predisposition to or the presence of autism or an autism spectrum disorder in a subject, said method comprising identifying the presence of absence of a genetic alteration in a JARID2 nucleic acid molecule derived from the subject.

2. The method of claim 1, wherein the subject is identified as having a family member diagnosed with autism.

3. The method of claim 1, wherein the subject is identified as having a family member diagnosed with schizophrenia.

4. The method of claim 1, wherein the genetic alteration is in a linkage disequilibrium region of JARID2 or is associated with chromosome 6p23.

5. The method of claim 1, wherein the genetic alteration is a single nucleotide polymorphism (SNP) in said JARID2 nucleic acid molecule.

6. The method of claim 5, wherein the SNP is selected from the group consisting of rs7766973 (SEQ ID NO: 2), rs6459404 (SEQ ID NO: 10), rs6921502 (SEQ ID NO: 8), rs6915344 (SEQ ID NO: 3), and rs13193457 (SEQ ID NO: 15).

7. The method of claim 5, wherein the identification of a C at polymorphism site rs7766973 (SEQ ID NO: 2), indicates an increased risk for autism.

8. The method of claim 1, wherein the genetic alteration is identified in a biological sample from the subject.

9. The method of claim 8, wherein the biological sample is selected from the group consisting of blood, urine, feces, saliva, a cheek swab, amniotic fluid, and tissue.

10. The method of claim 1, wherein the biological sample is blood.

11. The method of claim 1, wherein the sample is isolated from a subject that is between 0 and 6 months of age, between 6 and 12 months of age, or between 12 and 36 months of age.

12. The method of claim 1, wherein the subject is a child identified as having delayed communication skills, social skills, or that is otherwise identified as developmentally disabled.

13. The method of claim 1, wherein the method further comprises comparing the genetic alteration in the subject with a corresponding sequence in a relative of the subject.

14. The method of claim 1, wherein the genetic alteration is detected by a method selected from the group consisting of direct sequencing, single strand polymorphism assay, denaturing high performance liquid chromatography, hybridization on a nucleic acid array, restriction length polymorphism assay, ligase chain reaction, enzymatic cleavage, southern hybridization, mass spectrometry, and polymerase chain reaction.

15. The method of claim 1, wherein the biological sample comprises deoxyribonucleic acid or ribonucleic acid.

16. The method of claim 1, wherein the genetic alteration is detected by single strand polymorphism assay.

17. The method of claim 1, wherein the genetic alteration is detected using denaturing high performance liquid chromatography.

18. The method of claim 1, wherein the testing of the sample is carried out by direct sequencing of nucleic acids.

19. The method of claim 1, wherein the polymorphism is at a site selected from the group consisting of rs7766973 (SEQ ID NO: 2), rs6915344 (SEQ ID NO: 3), rs12530202 (SEQ ID NO: 4), rs2295954 (SEQ ID NO: 5), rs9464779 (SEQ ID NO: 6), rs11962776 (SEQ ID NO: 7), rs6921502 (SEQ ID NO: 8), rs9396578 (SEQ ID NO: 9), rs6459404 (SEQ ID NO: 10), rs9370809 (SEQ ID NO: 11), rs3759 (SEQ ID NO: 12), rs957387, (SEQ ID NO: 13), rs707833 (SEQ ID NO: 14), rs13193457 (SEQ ID NO: 15), rs909626 (SEQ ID NO: 16).

20. The method of claim 1, further comprising identifying the subject as having a developmental delay or behavioral abnormality characteristic of autism.

21. A method for identifying a subject as in need of therapeutic intervention to ameliorate autism or an autism spectrum disorder, the method comprising identifying the presence or absence of a genetic alteration in a JARID2 nucleic acid molecule derived from the subject.

22. A kit for detecting an autism-associated polymorphism in a subject, the kit comprising at least one polynucleotide molecule capable of specifically binding or hybridizing to a polymorphism in a JARID2 nucleic acid molecule and directions for using the kit in the method of claim 1.

23. A kit for detecting an autism-associated polymorphism in a subject, the kit comprising at least one set of primers suitable for use in polymerase chain reaction (PCR), wherein the set of primers amplifies a JARID2 nucleic acid molecule.

24. A kit for detecting an autism associated polymorphism in a subject, the kit comprising at least one set of primers suitable for use in polymerase chain reaction (PCR), wherein the set of primers amplifies polymorphism site selected from the group consisting of rs7766973 (SEQ ID NO: 2), rs6915344 (SEQ ID NO: 3), rs12530202 SEQ ID NO: 4), rs2295954 (SEQ ID NO: 5), rs9464779 (SEQ ID NO: 6), rs11962776 (SEQ ID NO: 7), rs6921502 (SEQ ID NO: 8), rs9396578 (SEQ ID NO: 9), rs6459404 (SEQ ID NO: 10), rs9370809 (SEQ ID NO: 11), rs3759 (SEQ ID NO: 12), rs957387 (SEQ ID NO: 13), rs707833, (SEQ ID NO: 14), rs13193457 (SEQ ID NO: 15), rs909626 (SEQ ID NO: 16).
